SEQUENCE LISTING (114 sequences)

SEQ ID NO: 1 - protein, 13 aa, Homo sapiens
RGQVRKLEKMTKR

SEQ ID NO: 2 - protein, 7 aa, Homo sapiens
ENVVTYV

SEQ ID NO: 3 - protein, 4 aa, Homo sapiens
YGAS

SEQ ID NO: 4 - protein, 7 aa, Homo sapiens
GQGYSYP

SEQ ID NO: 5 - protein, 8 aa, Homo sapiens
GYTFNTYA

SEQ ID NO: 6 - protein, 10 aa, Homo sapiens
IRSKSYNYVT

SEQ ID NO: 7 - protein, 9 aa, Homo sapiens
VKGNGNYVS

SEQ ID NO: 8 - protein, 107 aa, Mus musculus
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 9 - DNA, 321 bp, Mus musculus
  source 1..321: mol_type = other DNA
aacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaa

SEQ ID NO: 10 - protein, 121 aa, Mus musculus
EVQLVESGGGLVQPKGSLKLSCAASGYTFNTYAVNWVRQAPGKGLEWVARIRSKSYNYVTYYADSVKDRFTISRDDSQSMLYLQMNNLKTEDTAMYYCVKGNGNYVSFAYWGQGTLVTVSA

SEQ ID NO: 11 - DNA, 363 bp, Mus musculus
  source 1..363: mol_type = other DNA
gaggtgcagcttgttgagtctggtggaggattggtacagcctaaagggtcattgaaactctcatgtgcagcctctggatacaccttcaatacctacgccgtgaactgggtccgccaggctccaggaaagggtttggagtgggttgctcgcataagaagtaaaagttataattatgtaacatattatgccgattcagtgaaagacaggttcaccatctccagagatgattcacaaagcatgctctatctgcaaatgaacaacttgaaaactgaggacacagccatgtattactgtgtgaaaggaaatggtaactacgtttcgtttgcttactggggccaagggactctggtcactgtctctgca

SEQ ID NO: 12 - protein, 233 aa, Mus musculus
MESQTLVFISILLWLYGADGNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - DNA, 699 bp, Mus musculus
  source 1..699: mol_type = other DNA
atggagagccagaccctggtgttcatcagcatcctgctgtggctgtacggcgccgacggcaacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 14 - protein, 470 aa, Mus musculus
MTLNMLLGLKWVFFVVFYQGVHCEVQLVESGGGLVQPKGSLKLSCAASGYTFNTYAVNWYRQAPGKGLEWVARIRSKSYNYVTYYADSVKDRFTISRDDSQSMLYLQMNNLKTEDTAMYYCVKGNGNYVSFAYWGQGTLVTVSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIPRGPTIKPCPPCKCPPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 15 - DNA, 1407 bp, Mus musculus
  source 1..1407: mol_type = other DNA
atgaccctgaacatgctgctgggcctgaagtgggtgttcttcgtggtgttctaccagggcgtgcactgcgaggtgcagcttgttgagtctggtggaggattggtacagcctaaagggtcattgaaactctcatgtgcagcctctggatacaccttcaatacctacgccgtgaactgggtccgccaggctccaggaaagggtttggagtgggttgctcgcataagaagtaaaagttataattatgtaacatattatgccgattcagtgaaagacaggttcaccatctccagagatgattcacaaagcatgctctatctgcaaatgaacaacttgaaaactgaggacacagccatgtattactgtgtgaaaggaaatggtaactacgtttcgtttgcttactggggccaagggactctggtcactgtctctaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattcccagagggcccacaatcaagccctgtcctccatgcaaatgcccacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaatcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 16 - protein, 501 aa, Mus musculus
MDFQVQIFSFLLISASVIMSRGVHSEVQLVESGGGLVQPKGSLKLSCAASGYTFNTYAVNWVRQAPGKGLEWVARIRSKSYNYVTYYADSVKDRFTISRDDSQSMLYLQMNNLKTEDTAMYYCVKGNGNYVSFAYWGQGTLVTVSAGGGGSGGGGSGGGGSNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRAAAVPRDGGCKPCICTGTVPEVPSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGI

SEQ ID NO: 17 - DNA, 1503 bp, Mus musculus
  source 1..1503: mol_type = other DNA
atggattttcaggtgcagattttcagcttcctgctaatcagtgcctcagtcataatgtctagaggtgtccactccgaggtgcagcttgttgagtctggtggaggattggtacagcctaaagggtcattgaaactctcatgtgcagcctctggatacaccttcaatacctacgccgtgaactgggtccgccaggctccaggaaagggtttggagtgggttgctcgcataagaagtaaaagttataattatgtaacatattatgccgattcagtgaaagacaggttcaccatctccagagatgattcacaaagcatgctctatctgcaaatgaacaacttgaaaactgaggacacagccatgtattactgtgtgaaaggaaatggtaactacgtttcgtttgcttactggggccaagggactctggtcactgtctctgcaggaggtggtggatcgggcggtggcgggtcgggtggcggcggatctaacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaacgggcggccgctgtgccaagagatggcggctgcaagccctgtatctgcaccggaaccgtgcccgaagtgcccagcgtgttcatcttcccacctaagcctaaggacgtgctgaccatcacactgacccctaaagtgacctgcgtggtggtggacatctccaaggacgatcccgaggtgcagttcagttggttcgtggacgacgtggaagtgcacaccgctcagacacagcccagagaggaacagttcaacagcaccttcagaagcgtgtccgagctgcccatcatgcaccaggattggctgaacggcaaagaattcaagtgcagagtgaacagcgccgcctttcctgctcctatcgagaaaacaatcagcaagaccaagggcagacccaaggctccccaggtgtacacaatccctccacctaaagaacagatggccaaggacaaggtgtccctgacctgcatgatcaccgatttcttcccagaggacatcaccgtggaatggcagtggaacggacagcccgccgagaactacaagaacacacagcctatcatgaacaccaacggcagctacttcgtgtacagcaagctgaacgtgcagaagtccaactgggaggccggcaacaccttcacctgttctgtgctgcacgagggcctgcacaaccaccacacagagaagtccctgtctcacagccccggcatc

SEQ ID NO: 18 - protein, 504 aa, Homo sapiens
MDFQVQIFSFLLISASVIMSRGVHSEVQLVESGGGLVQPKGSLKLSCAASGYTFNTYAVNWVRQAPGKGLEWVARIRSKSYNYVTYYADSVKDRFTISRDDSQSMLYLQMNNLKTEDTAMYYCVKGNGNYVSFAYWGQGTLVTVSAGGGGSGGGGSGGGGSNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRAAAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1497 bp, Homo sapiens
  source 1..1497: mol_type = other DNA
atggattttcaggtgcagattttcagcttcctgctaatcagtgcctcagtcataatgtctagaggtgtccactccgaggtgcagcttgttgagtctggtggaggattggtacagcctaaagggtcattgaaactctcatgtgcagcctctggatacaccttcaatacctacgccgtgaactgggtccgccaggctccaggaaagggtttggagtgggttgctcgcataagaagtaaaagttataattatgtaacatattatgccgattcagtgaaagacaggttcaccatctccagagatgattcacaaagcatgctctatctgcaaatgaacaacttgaaaactgaggacacagccatgtattactgtgtgaaaggaaatggtaactacgtttcgtttgcttactggggccaagggactctggtcactgtctctggaggtggtggatcgggcggtggcgggtcgggtggcggcggatctaacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaagagcccaaatctcctgacaaaactcacacatgcccaccgtgcccagcacctgagctgctgggcggaccgtcagtcttcctcttccccccaaaacccaaggacaccctcatgatcgcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaagccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 20 - protein, 477 aa, Homo sapiens
MDFQVQIFSFLLISASVIMSRGVHSEVQLVESGGGLVQPKGSLKLSCAASGYTFNTYAVNWVRQAPGKGLEWVARIRSKSYNYVTYYADSVKDRFTISRDDSQSMLYLQMNNLKTEDTAMYYCGGGGSGGGGSGGGGSNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1431 bp, Homo sapiens
  source 1..1431: mol_type = other DNA
atggattttcaggtgcagattttcagcttcctgctaatcagtgcctcagtcataatgtctagaggtgtccactccgaggtgcagcttgttgagtctggtggaggattggtacagcctaaagggtcattgaaactctcatgtgcagcctctggatacaccttcaatacctacgccgtgaactgggtccgccaggctccaggaaagggtttggagtgggttgctcgcataagaagtaaaagttataattatgtaacatattatgccgattcagtgaaagacaggttcaccatctccagagatgattcacaaagcatgctctatctgcaaatgaacaacttgaaaactgaggacacagccatgtattactgtggaggtggtggatcgggcggtggcgggtcgggtggcggcggatctaacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaagagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaagccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 22 - protein, 68 aa, Homo sapiens
FWVLVVVGGVLACYSLLVTVAFIIFWVRSKRSRLLHSDYMNMTPRRPGPTRKHYQPYAPPRDFAAYRS

SEQ ID NO: 23 - DNA, 204 bp, Homo sapiens
  source 1..204: mol_type = other DNA
ttttgggtgctggtggtggttggtggagtcctggcttgctatagcttgctagtaacagtggcctttattattttctgggtgaggagtaagaggagcaggctcctgcacagtgactacatgaacatgactcccagacgccctggccccacccgcaagcattaccagccctatgcccccccacgcgacttcgcagcctatagatct

SEQ ID NO: 24 - protein, 113 aa, Homo sapiens
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - DNA, 339 bp, Homo sapiens
  source 1..339: mol_type = other DNA
ctgagagtgaagttcagcaggagcgcagacgcccccgcgtaccagcagggccagaaccagctctataacgaactcaatctaggacgaagagaggagtacgatgttttggacaagagacgtggccgggaccctgagatggggggaaagccgagaaggaagaaccctcaggagggcctgtataatgaactccagaaagataagatggcggaggcttacagtgagattgggatgaaaggcgagcgccggaggggcaaggggcacgatggcctttaccagggtctcagtacagccaccaaggacacctacgacgcccttcacatgcaggctctgcctcctagg

SEQ ID NO: 26 - protein, 36 aa, Homo sapiens
RDQRLPPDAHKPPGGGSFRTPIQEEQADAHSTLAKI

SEQ ID NO: 27 - DNA, 108 bp, Homo sapiens
  source 1..108: mol_type = other DNA
cgggaccagaggctgcctcccgatgctcacaagccccctggaggaggcagtttcaggacccccatccaagaggagcaggccgacgcccactccaccctggctaaaatc

SEQ ID NO: 28 - protein, 694 aa, synthetic construct
MDFQVQIFSFLLISASVIMSRGVHSEVQLVESGGGLVQPKGSLKLSCAASGYTFNTYAVNWVRQAPGKGLEWVARIRSKSYNYVTYYADSVKDRFTISRDDSQSMLYLQMNNLKTEDTAMYYCGGGGSGGGGSGGGGSNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRDQRLPPDAHKPPGGGSFRTPIQEEQADAHSTLAKI

SEQ ID NO: 29 - DNA, 2082 bp, synthetic construct
  source 1..2082: mol_type = other DNA
atggattttcaggtgcagattttcagcttcctgctaatcagtgcctcagtcataatgtctagaggtgtccactccgaggtgcagcttgttgagtctggtggaggattggtacagcctaaagggtcattgaaactctcatgtgcagcctctggatacaccttcaatacctacgccgtgaactgggtccgccaggctccaggaaagggtttggagtgggttgctcgcataagaagtaaaagttataattatgtaacatattatgccgattcagtgaaagacaggttcaccatctccagagatgattcacaaagcatgctctatctgcaaatgaacaacttgaaaactgaggacacagccatgtattactgtggaggtggtggatcgggcggtggcgggtcgggtggcggcggatctaacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaagagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaagccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaattttgggtgctggtggtggttggtggagtcctggcttgctatagcttgctagtaacagtggcctttattattttctgggtgaggagtaagaggagcaggctcctgcacagtgactacatgaacatgactcccagacgccctggccccacccgcaagcattaccagccctatgcccccccacgcgacttcgcagcctatagatctctgagagtgaagttcagcaggagcgcagacgcccccgcgtaccagcagggccagaaccagctctataacgaactcaatctaggacgaagagaggagtacgatgttttggacaagagacgtggccgggaccctgagatggggggaaagccgagaaggaagaaccctcaggagggcctgtataatgaactccagaaagataagatggcggaggcttacagtgagattgggatgaaaggcgagcgccggaggggcaaggggcacgatggcctttaccagggtctcagtacagccaccaaggacacctacgacgcccttcacatgcaggctctgcctcctaggcgggaccagaggctgcctcccgatgctcacaagccccctggaggaggcagtttcaggacccccatccaagaggagcaggccgacgcccactccaccctggctaaaatc

SEQ ID NO: 30 - protein, 1397 aa, Homo sapiens
MEEISAAAVKVVPGPERPSPFSQLVYTSNDSYIVHSGDLRKIHKAASRGQVRKLEKMTKRKKTINLNIQDAQKRTALHWACVNGHEEVVTFLVDRKCQLDVLDGEHRTPLMKALQCHQEACANILIDSGADINLVDVYGNTALHYAVYSEILSVVAKLLSHGAVIEVHNKASLTPLLLSITKRSEQIVEFLLIKNANANAVNKYKCTALMLAVCHGSSEIVGMLLQQNVDVFAADICGVTAEHYAVTCGFHHIHEQIMEYIRKLSKNHQNTNPEGTSAGTPDEAAPLAERTPDTAESLVEKTPDEAAPLVERTPDTAESLVEKTPDEAASLVEGTSDKIQCLEKATSGKFEQSAEETPREITSPAKETSEKFTWPAKGRPRKIAWEKKEDTPREIMSPAKETSEKFTWAAKGRPRKIAWEKKETPVKTGCVARVTSNKTKVLEKGRSKMIACPTKESSTKASANDQRFPSESKQEEDEEYSCDSRSLFESSAKIQVCIPESIYQKVMEINREVEEPPKKPSAFKPAIEMQNSVPNKAFELKNEQTLRADPMFPPESKQKDYEENSWDSESLCETVSQKDVCLPKATHQKEIDKINGKLEESPNKDGLLKATCGMKVSIPTKALELKDMQTFKAEPPGKPSAFEPATEMQKSVPNKALELKNEQTLRADEILPSESKQKDYEENSWDTESLCETVSQKDVCLPKAAHQKEIDKINGKLEGSPVKDGLLKANCGMKVSIPTKALELMDMQTFKAEPPEKPSAFEPAIEMQKSVPNKALELKNEQTLRADEILPSESKQKDYEESSWDSESLCETVSQKDVCLPKATHQKEIDKINGKLEESPDNDGFLKAPCRMKVSIPTKALELMDMQTFKAEPPEKPSAFEPAIEMQKSVPNKALELKNEQTLRADQMFPSESKQKKVEENSWDSESLRETVSQKDVCVPKATHQKEMDKISGKLEDSTSLSKILDTVHSCERARELQKDHCEQRTGKMEQMKKKFCVLKKKLSEAKEIKSQLENQKVKWEQELCSVRLTLNQEEEKRRNADILNEKIREELGRIEEQHRKELEVKQQLEQALRIQDIELKSVESNLNQVSHTHENENYLLHENCMLKKEIAMLKLEIATLKHQYQEKENKYFEDIKILKEKNAELQMTLKLKEESLTKRASQYSGQLKVLIAENTMLTSKLKEKQDKEILEAEIESHHPRLASAVQDHDQIVTSRKSQEPAFHIAGDACLQRKMNVDVSSTIYNNEVLHQPLSEAQRKSKSLKINLNYAGDALRENTLVSEHAQRDQRETQCQMKEAEHMYQNEQDNVNKHTEQQESLDQKLFQLQSKNMWLQQQLVHAHKKADNKSKITIDIHFLERKMQHHLLKEKNEEIFNYNNHLKNRIYQYEKEKAETENS

SEQ ID NO: 31 - protein, 1392 aa, Homo sapiens
MKRLLAAAGKGVRGPEPPNPFSERVYTEKDYGTIYFGDLGKIHTAASRGQVQKLEKMTVGKKPVNLNKRDMKKRTALHWACVNGHAEVVTFLVDRKCQLNVLDGEGRTPLMKALQCEREACANILIDAGADLNYVDVYGNTALHYAVYSENLLMVATLLSYGAVIEVQNKASLTPLLLAIQKRSKQTVEFLLTKNANANAFNESKCTALMLAICEGSSEIVGMLLQQNVDVFAEDIHGITAERYAAACGVNYIHQQLLEHIRKLPKNPQNTNPEGTSTGTPDEAAPLAERTPDTAESLLEKTPDEAARLVEGTSAKIQCLGKATSGKFEQSTEETPRKILRPTKETSEKFSWPAKERSRKITWEEKETSVKTECVAGVTPNKTEVLEKGTSNMIACPTKETSTKASTNVDVSSVEPIFSLFGTRTIENSQCTKVEEDFNLATKIISKSAAQNYTCLPDATYQKDIKTINHKIEDQMFPSESKREEDEEYSWDSGSLFESSAKTQVCIPESMYQKVMEINREVEELPEKPSAFKPAVEMQKTVPNKAFELKNEQTLRAAQMFPSESKQKDDEENSWDSESPCETVSQKDVYLPKATHQKEFDTLSGKLEESPVKDGLLKPTCGRKVSLPNKALELKDRETFKAESPDKDGLLKPTCGRKVSLPNKALELKDRETLKAESPDNDGLLKPTCGRKVSLPNKALELKDRETFKAAQMFPSESKQKDDEENSWDFESFLETLLQNDVCLPKATHQKEFDTLSGKLEESPDKDGLLKPTCGMKISLPNKALELKDRETFKAEDVSSVESTFSLFGKPTTENSQSTKVEEDFNLTTKEGATKTVTGQQERDIGIIERAPQDQTNKMPTSELGRKEDTKSTSDSEIISVSDTQNYECLPEATYQKEIKTTNGKIEESPEKPSHFEPATEMQNSVPNKGLEWKNKQTLRADSTTLSKILDALPSCERGRELKKDNCEQITAKMEQTKNKFCVLQKELSEAKEIKSQLENQKAKWEQELCSVRLTLNQEEEKRRNVDILKEKIRPEEQLRKKLEVKQQLEQTLRIQDIELKSVTSNLNQVSHTHESENDLFHENCMLKKEIAMLKLEVATLKHQHQVKENKYFEDIKILQEKNAELQMTLKLKQKTVTKRASQYREQLKVLTAENTMLTSKLKEKQDKEILETEIESHHPRLASALQDHDQSVTSRKNQELAFHSAGDAPLQGIMNVDVSNTIYNNEVLHQPLYEAQRKSKSPKINLNYAGDDLRENALVSEHAQRDRCETQCQMKKAEHMYQNEQDNVDKHTEQQESLEQKLFQLESKNRWLRQQLVYAHKKVNKSKVTINIQFPEMKMQRHLNEKNEEVFNYGNHLKERIDQYEKEKAEREVSIKKYKYFSNFLKESGLG

SEQ ID NO: 32 - protein, 619 aa, Homo sapiens
MKRLLAAAGKGVRGPEPPNPFSERVYTEKDYGTIYFGDLGKIHTAASRGQVQKLEKMTVGKKPVNLNKRDMKKRTALHWACVNGHAEVVTFLVDRKCQLNVLDGEGRTPLMKALQCEREACANILIDAGADLNYVDVYGNTALHYAVYSENLLMVATLLSYGAVIEVQNKASLTPLLLAIQKRSKQTVEFLLTKNANANAFNESKCTALMLAICEGSSEIVGMLLQQNVDVFAEDIHGITAERYAAACGVNYIHQQLLEHIRKLPKNPQNTNPEGTSTGTPDEAAPLAERTPDTAESLLEKTPDEAARLVEGTSAKIQCLGKATSGKFEQSTEETPRKILRPTKETSEKFSWPAKERSRKITWEEKETSVKTECVAGVTPNKTEVLEKGTSNMIACPTKETSTKASTNVDVSSVEPIFSLFGTRTIENSQCTKVEEDFNLATKIISKSAAQNYTCLPDATYQKDIKTINHKIEDQMFPSESKREEDEEYSWDSGSLFESSAKTQVCIPESMYQKVMEINREVEELPEKPSAFKPAVEMQKTVPNKAFELKNEQTLRAAQMFPSESKQKDDEENSWDSESPCETVSQKDVYLPKATHQKEFDTLSGKLEAYLWKESFSSK

SEQ ID NO: 33 - protein, 13 aa, Homo sapiens
LKNEQTLRADQMF

SEQ ID NO: 34 - protein, 703 aa, Mus musculus
MESQTLVFISILLWLYGADGNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECMTLNMLLGLKWVFFVVFYQGVHCEVQLVESGGGLVQPKGSLKLSCAASGYTFNTYAVNWYRQAPGKGLEWVARIRSKSYNYVTYYADSVKDRFTISRDDSQSMLYLQMNNLKTEDTAMYYCVKGNGNYVSFAYWGQGTLVTVSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIPRGPTIKPCPPCKCPPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 35 - protein, 13 aa, Homo sapiens
CANILIDSGADIN

SEQ ID NO: 36 - protein, 13 aa, Homo sapiens
ACANILIDSGADI

SEQ ID NO: 37 - protein, 13 aa, Homo sapiens
EACANILIDSGAD

SEQ ID NO: 38 - protein, 13 aa, Homo sapiens
QEACANILIDSGA

SEQ ID NO: 39 - protein, 13 aa, Homo sapiens
HQEACANILIDSG

SEQ ID NO: 40 - protein, 13 aa, Homo sapiens
CHQEACANILIDS

SEQ ID NO: 41 - protein, 13 aa, Homo sapiens
QCHQEACANILID

SEQ ID NO: 42 - protein, 13 aa, Homo sapiens
LQCHQEACANILI

SEQ ID NO: 43 - protein, 13 aa, Homo sapiens
ALQCHQEACANIL

SEQ ID NO: 44 - protein, 13 aa, Homo sapiens
KALQCHQEACANI

SEQ ID NO: 45 - protein, 13 aa, Homo sapiens
MKALQCHQEACAN

SEQ ID NO: 46 - protein, 13 aa, Homo sapiens
LMKALQCHQEACA

SEQ ID NO: 47 - protein, 13 aa, Homo sapiens
PLMKALQCHQEAC

SEQ ID NO: 48 - protein, 13 aa, Homo sapiens
TPLMKALQCHQEA

SEQ ID NO: 49 - protein, 13 aa, Homo sapiens
RTPLMKALQCHQE

SEQ ID NO: 50 - protein, 13 aa, Homo sapiens
HRTPLMKALQCHQ

SEQ ID NO: 51 - protein, 13 aa, Homo sapiens
EHRTPLMKALQCH

SEQ ID NO: 52 - protein, 13 aa, Homo sapiens
GEHRTPLMKALQC

SEQ ID NO: 53 - protein, 13 aa, Homo sapiens
DGEHRTPLMKALQ

SEQ ID NO: 54 - protein, 13 aa, Homo sapiens
LDGEHRTPLMKAL

SEQ ID NO: 55 - protein, 13 aa, Homo sapiens
VLDGEHRTPLMKA

SEQ ID NO: 56 - protein, 13 aa, Homo sapiens
DVLDGEHRTPLMK

SEQ ID NO: 57 - protein, 13 aa, Homo sapiens
LDVLDGEHRTPLM

SEQ ID NO: 58 - protein, 13 aa, Homo sapiens
QLDVLDGEHRTPL

SEQ ID NO: 59 - protein, 13 aa, Homo sapiens
CQLDVLDGEHRTP

SEQ ID NO: 60 - protein, 13 aa, Homo sapiens
KCQLDVLDGEHRT

SEQ ID NO: 61 - protein, 13 aa, Homo sapiens
RKCQLDVLDGEHR

SEQ ID NO: 62 - protein, 13 aa, Homo sapiens
DRKCQLDVLDGEH

SEQ ID NO: 63 - protein, 13 aa, Homo sapiens
VDRKCQLDVLDGE

SEQ ID NO: 64 - protein, 13 aa, Homo sapiens
LVDRKCQLDVLDG

SEQ ID NO: 65 - skipped

SEQ ID NO: 66 - protein, 13 aa, Homo sapiens
TFLVDRKCQLDVL

SEQ ID NO: 67 - protein, 13 aa, Homo sapiens
VTFLVDRKCQLDV

SEQ ID NO: 68 - protein, 13 aa, Homo sapiens
VVTFLVDRKCQLD

SEQ ID NO: 69 - protein, 13 aa, Homo sapiens
EVVTFLVDRKCQL

SEQ ID NO: 70 - protein, 13 aa, Homo sapiens
EEVVTFLVDRKCQ

SEQ ID NO: 71 - protein, 13 aa, Homo sapiens
HEEVVTFLVDRKC

SEQ ID NO: 72 - protein, 13 aa, Homo sapiens
GHEEVVTFLVDRK

SEQ ID NO: 73 - protein, 13 aa, Homo sapiens
NGHEEVVTFLVDR

SEQ ID NO: 74 - protein, 13 aa, Homo sapiens
VNGHEEVVTFLVD

SEQ ID NO: 75 - protein, 13 aa, Homo sapiens
CVNGHEEVVTFLV

SEQ ID NO: 76 - protein, 13 aa, Homo sapiens
ACVNGHEEVVTFL

SEQ ID NO: 77 - protein, 13 aa, Homo sapiens
WACVNGHEEVVTF

SEQ ID NO: 78 - protein, 13 aa, Homo sapiens
HWACVNGHEEVVT

SEQ ID NO: 79 - protein, 13 aa, Homo sapiens
LHWACVNGHEEVV

SEQ ID NO: 80 - protein, 13 aa, Homo sapiens
ALHWACVNGHEEV

SEQ ID NO: 81 - protein, 13 aa, Homo sapiens
TALHWACVNGHEE

SEQ ID NO: 82 - protein, 13 aa, Homo sapiens
RTALHWACVNGHE

SEQ ID NO: 83 - protein, 13 aa, Homo sapiens
KRTALHWACVNGH

SEQ ID NO: 84 - protein, 13 aa, Homo sapiens
QKRTALHWACVNG

SEQ ID NO: 85 - protein, 13 aa, Homo sapiens
AQKRTALHWACVN

SEQ ID NO: 86 - protein, 13 aa, Homo sapiens
DAQKRTALHWACV

SEQ ID NO: 87 - protein, 13 aa, Homo sapiens
QDAQKRTALHWAC

SEQ ID NO: 88 - protein, 13 aa, Homo sapiens
IQDAQKRTALHWA

SEQ ID NO: 89 - protein, 13 aa, Homo sapiens
NIQDAQKRTALHW

SEQ ID NO: 90 - protein, 13 aa, Homo sapiens
LNIQDAQKRTALH

SEQ ID NO: 91 - protein, 13 aa, Homo sapiens
NLNIQDAQKRTAL

SEQ ID NO: 92 - protein, 13 aa, Homo sapiens
INLNIQDAQKRTA

SEQ ID NO: 93 - protein, 13 aa, Homo sapiens
TINLNIQDAQKRT

SEQ ID NO: 94 - protein, 13 aa, Homo sapiens
KTINLNIQDAQKR

SEQ ID NO: 95 - protein, 13 aa, Homo sapiens
KKTINLNIQDAQK

SEQ ID NO: 96 - protein, 13 aa, Homo sapiens
RKKTINLNIQDAQ

SEQ ID NO: 97 - protein, 13 aa, Homo sapiens
KRKKTINLNIQDA

SEQ ID NO: 98 - protein, 13 aa, Homo sapiens
TKRKKTINLNIQD

SEQ ID NO: 99 - protein, 13 aa, Homo sapiens
MTKRKKTINLNIQ

SEQ ID NO: 100 - protein, 13 aa, Homo sapiens
KMTKRKKTINLNI

SEQ ID NO: 101 - protein, 13 aa, Homo sapiens
EKMTKRKKTINLN

SEQ ID NO: 102 - protein, 13 aa, Homo sapiens
LEKMTKRKKTINL

SEQ ID NO: 103 - protein, 13 aa, Homo sapiens
KLEKMTKRKKTIN

SEQ ID NO: 104 - protein, 13 aa, Homo sapiens
RKLEKMTKRKKTI

SEQ ID NO: 105 - protein, 13 aa, Homo sapiens
VRKLEKMTKRKKT

SEQ ID NO: 106 - protein, 13 aa, Homo sapiens
QVRKLEKMTKRKK

SEQ ID NO: 107 - protein, 13 aa, Homo sapiens
GQVRKLEKMTKRK

SEQ ID NO: 108 - protein, 13 aa, Homo sapiens
SRGQVRKLEKMTK

SEQ ID NO: 109 - protein, 13 aa, Homo sapiens
ASRGQVRKLEKMT

SEQ ID NO: 110 - protein, 13 aa, Homo sapiens
AASRGQVRKLEKM

SEQ ID NO: 111 - protein, 13 aa, Homo sapiens
KAASRGQVRKLEK

SEQ ID NO: 112 - protein, 13 aa, Homo sapiens
HKAASRGQVRKLE

SEQ ID NO: 113 - protein, 13 aa, Homo sapiens
IHKAASRGQVRKL

SEQ ID NO: 114 - protein, 13 aa, Homo sapiens
KIHKAASRGQVRK

(19) United States
(12) Patent Application Publication
(10) Pub. No.: US 2026/0139072 A1
(43) Pub. Date: May 21, 2026

(54) IMMUNOREACTIVE MOLECULES AND USES THEREOF

(71) Applicants: UNIVERSITAET HEIDELBERG, Heidelberg (DE); DEUTSCHES KREBSFORSCHUNGSZENTRUM STIFTUNG DES OEFFENTLICHEN RECHTS, Heidelberg (DE)

(72) Inventors: Patrick Schmidt, Heidelberg (DE); Inka ZOERNIG, Frankfurt (DE); Dirk JAEGER, Heidelberg (DE); Frank MOMBURG, Heidelberg (DE); Aileen BERGER, Breuberg (DE)

(73) Assignees: UNIVERSITAET HEIDELBERG, Heidelberg (DE); DEUTSCHES KREBSFORSCHUNGSZENTRUM STIFTUNG DES OEFFENTLICHEN RECHTS, Heidelberg (DE)

(21) Appl. No.: 18/707,751

(22) PCT Filed: Nov. 10, 2022
(86) PCT No.: PCT/EP2022/081522

§ 371 Date: May 6, 2024

(30) Foreign Application Priority Data
Nov. 11, 2021 (EP) 21207816.6

(51) Int. Cl. C07K 16/30; A61P 35/00; G01N 33/575

(52) U.S. Cl. CPC C07K 16/3015; A61P 35/00; G01N 33/5758; C07K 2317/33; C07K 2317/52; C07K 2317/622

(57) ABSTRACT

The present invention relates to an immunoreactive molecule that specifically recognises and binds to human Ankyrin Repeat Domain-Containing Protein 30A (NY-BR-1) and, in particular, to an immunoreactive molecule, which shows no cross-reactivity to other human Ankyrin repeat domain containing proteins. The invention further relates to the use of such immunoreactive molecules in the treatment of cancer as well as in companion diagnostics methods.

### FIELD OF THE INVENTION

The present invention relates to an immunoreactive molecule that specifically recognises and binds to human Ankyrin Repeat Domain-Containing Protein 30A (NY-BR-1) and, in particular, to an immunoreactive molecule, which shows no cross-reactivity to other human ankyrin repeat domain containing proteins. The invention further relates to the use of such immunoreactive molecules in the treatment of cancer as well as in companion diagnostics methods and will be described hereinafter with reference to these applications. However, it will be appreciated that the invention is not limited to this particular field of use.

### BACKGROUND OF THE INVENTION

Any discussion of the background art throughout the specification should in no way be considered as an admission that such art is widely known or forms part of common general knowledge in the field.

In the context of cancer therapy, immunotherapy typically aims at eliciting an immune response targeting tumour cells. Immunoreactive molecules, i.e. molecules that specifically recognise and bind a target sequence or epitope of an antigen of a tumour cell, are required to initiate the immune response and therefore constitute an integral tool for an immunotherapeutic approach in the treatment of cancer. Immunoreactive molecules, such as monoclonal antibodies against a tumour-specific antigen (i.e. a “cancer antigen”; also referred to as “tumour-associated antigen” or “cancer associated antigen” below), have become part of many cancer treatment regimens. For example, the monoclonal antibody trastuzumab is approved for the treatment of breast and stomach cancers as it specifically targets an epitope of the HER2 receptor. However, aside from “regular” antibodies targeting cancer antigens, several different formats of immunoreactive molecules have since been genetically engineered, which allow for the utilisation of their target-specificity in order to elicit an immune response. For example, chimeric antigen receptor (CAR) molecules constitute such an alternative format because they are fusion proteins, which combine an antibody-derived antigen recognition domain with, at least, a T-cell activation domain and, typically, a trans-membrane domain. This allows for the ex vivo production of patient-derived but genetically engineered T-cells carrying CARs with their antigen recognition domain positioned in the extracellular space, while the T-cell activation domain is positioned intracellularly, which again serve as patient-specific, tumour-specific and genetically-engineered, cellular anti-cancer agents. Cellular immunotherapies with such cellular anti-cancer agents provide a new option in the treatment of cancer patients—in particular of leukaemia patients-through reinfusion of the engineered T-cells into the patient.

Since this method was first described in the 1990s, and especially since clinical successes in 2011, this type of immunotherapy has crystallized as superior to standard treatment methods. More than 600 active phase I and II clinical trials have already been registered worldwide evaluating CAR T-cells. With respect to childhood and adult acute lymphoblastic leukemias (ALL) response rates of 80% on average have been reported in several completed studies. The majority of these include long-lasting complete remissions. With respect to application of the concept for the treatment of solid tumours, however, only a few clinical results have been reported and only relatively little clinical data is available. However, initial success has been reported in glioblastoma patients. The main reason for this discrepancy is likely the heterogeneous expression patterns of tumour-associated antigens in solid tissues and/or minimal target antigen expression also on healthy tissue leading to cross-reactivity between tumour and healthy tissue. As will be understood, cross-reactivity of a CAR T-cell with healthy tissue may have detrimental consequences in a clinical context, as CAR T-cells do not discriminate between healthy and tumour cells. Accordingly, healthy cells—even if only weakly expressing the tumour-associated antigen—will equally be targeted.

NY-BR-1 belongs to the group of cancer testis antigens, which-due to their naturally restricted expression pattern (i.e. only in testis)—have been considered to be particularly well-suited as targets for NY-BR-1-specific CAR T-cells. However, NY-BR-1 was also identified as being associated with breast cancer (Theurillat et al. “NY-BR-1 protein expression in breast carcinoma: a mammary gland differentiation antigen as target for cancer immunotherapy” Cancer Immunol Immunother 2007 November; 56 (11): 1723-31) and as a potential target for antibody-based therapies of breast cancer (Seil et al. “The differentiation antigen NY-BR-1 is a potential target for antibody-based therapies in breast cancer” Int J Cancer 2007 Jun. 15; 120 (12): 2635-42). Seil et al. 2007 also published a number of anti-NY-BR-1 antibodies of which “Clone2” was shown to be particularly promising. Clone2 is the only anti-NY-BR-1 antibody commercially available and routinely used in published R&D efforts. For example, Clone2 is described as the primary antibody used for the detection of NY-BR-1 in western blot analysis in Das et al. 2019 (Das et al. “A transplantable tumour model allowing investigation of NY-BR-1-specific T cell responses in HLA-DRB1*0401 transgenic mice” BMC Cancer 2019 Sep. 13; 19 (1): 914). Through epitope analysis, the present inventors found that the specific binding sequence recognised by the Clone2 antibody is near-identical to an amino acid sequence (only different in a single amino acid) of NY-BR1.1 and its Isoform 2-two closely related proteins. Database and literature research revealed that NY-BR1.1 is also expressed to a significant extent in healthy brain tissue. As such, use of a Clone2 antibody derived CAR construct bears a great risk of also recognising NY-BR1.1 and its Isoform 2 in healthy tissues, thereby typically ruling such constructs out for the treatment of breast cancer patients.

There is a need in the art for improved immunoreactive molecules targeting NY-BR-1 such as to increase therapeutic options in the treatment of cancer, in particular testicular and breast cancer.

It is an object of the present invention to overcome or ameliorate at least one of the disadvantages of the prior art, or to provide a useful alternative. In particular, it is an object of the present invention to provide improved immunoreactive molecules targeting NY-BR-1.

### SUMMARY OF THE INVENTION

As indicated above, the present invention aims at providing immunoreactive molecules, which are highly specific for human Ankyrin Repeat Domain-Containing Protein 30A (NY-BR-1; UniProtKB Q9BXX3) but which, importantly, show no cross-reactivity to other human Ankyrin repeat domain containing proteins, such as human Ankyrin Repeat Domain-Containing Protein 30B (NY-BR-1.1; UniProtKB Q9BXX2) or Isoform 2 thereof (NY-BR-1.1 Isoform 2; UniProtKB Q9BXX2-2).

The immunoreactive molecules of the present invention are particularly useful in new cancer treatment regimens as well as in NY-BR-1 detection methods such as NY-BR-1 detection methods applied as companion diagnostics (CDx) methods during a patient's cancer treatment.

Broadly, the present disclosure relates to an immunoreactive molecule, wherein said immunoreactive molecule specifically recognises and binds to human Ankyrin Repeat Domain-Containing Protein 30A (NY-BR-1; UniProtKB Q9BXX3; SEQ ID NO:30) characterised in that the immunoreactive molecule shows no cross-reactivity to human Ankyrin Repeat Domain-Containing Protein 30B (NY-BR-1.1; UniProtKB Q9BXX2; SEQ ID NO:31) or Isoform 2 thereof (NY-BR-1.1 Isoform 2; UniProtKB Q9BXX2-2; SEQ ID NO:32). Preferably, the immunoreactive molecule specifically recognises and binds to an epitope of NY-BR-1 comprising the antigen peptide sequence RGQVRKLEKMTKR (SEQ ID NO. 1).

Specifically, in a first aspect, the present invention relates to an immunoreactive molecule, wherein said immunoreactive molecule specifically recognises and binds to human Ankyrin Repeat Domain-Containing Protein 30A (NY-BR-1; UniProtKB Q9BXX3) characterised in that the immunoreactive molecule specifically recognises and binds to an epitope of NY-BR-1 comprising the antigen peptide sequence of SEQ ID NO:1 and wherein said immunoreactive molecule has an antigen binding region comprising V_{L} complementarity-determining regions (CDRs) 1 to 3 of SEQ ID NOs: 2 to 4 and V_{H} CDRs 1 to 3 of SEQ ID NOs: 5 to 7.

In a second aspect, the present invention relates the immunoreactive molecules of the first aspect for use in the treatment of cancer.

Accordingly, this second aspect of the present invention also encompasses methods of treating cancer, wherein the method comprises administering the immunoreactive molecules of the first aspect to a subject in need thereof and/or use of an immunoreactive molecule of the first aspect in the manufacture of a medicament for the treatment of cancer.

Similarly, this second aspect also encompasses the use of the immunoreactive molecules of the first aspect in the preparation of a medicament for the treatment of cancer.

Typically, the cancer is characterised by the expression of the cancer associated antigen NY-BR-1, such as, without limitation, testicular cancer or breast cancer.

In a third aspect, the present invention relates to use of the immunoreactive molecule of the first aspect in a method of detecting NY-BR-1 in a sample, wherein, optionally, the sample is a sample previously obtained and/or derived from a cancer patient and the method is a companion diagnostics (CDx) method.

Preferably, the method is selected from the group consisting of: immunohistochemical staining methods; membrane-mediated protein blotting methods; and flow cytometry-based methods, and/or wherein the sample is a tissue sample, a primary cell sample or a cell line sample.

As such, in another embodiment, this third aspect, also encompasses such methods of detecting NY-BR-1 in a sample, comprising the use of the immunoreactive molecule of the first aspect.

### FIGURES

Embodiments of the invention will now be described, by way of example only, with reference to the accompanying drawings in which:

FIG. 1 in accordance with the experiments described in Example 1, illustrates the results of a flow cytometric analysis of NY-BR-1 and NY-BR-1.1 transfected Bosc23 cells using the primary murine full-length 10D11 antibody comprising SEQ ID NO: 12 and SEQ ID NO: 14 at concentrations of 2.5-50 μg/mL or the “Clone2” antibody at a concentration of 5 g/mL antibodies (ud=undiluted, 1:10-1:500 diluted), followed by incubation with the secondary Phycoerythrin (PE)-conjugated anti-mouse Fcγ subclass I specific antibody.

FIG. 2 in accordance with the experiments described in Example 2, illustrates the results of a Type 1 Mapping of monoclonal antibody 10D11 of SEQ ID NO: 34 on a microarray spotted in duplicates with overlapping 13aa peptides of NY-BR-1. Detection of bound 10D11 antibody of SEQ ID: 34 was performed using a polyclonal goat anti-mouse IgG(H+L) conj. DyLight680 antibody.

FIG. 3. in accordance with the experiments described in Example 3, illustrates the results of a flow cytometric analysis of NY-BR-1 and NY-BR-1.1 transfected Bosc23 cells using the 10D11-derived scFv-human IgG1-Fc fusion protein of SEQ ID NO:18 or the corresponding Clone2-derived scFv-human IgG1-Fc fusion protein, followed by incubation with the secondary Phycoerythrin (PE)—conjugated anti-human Fcγ fragment specific antibody.

FIG. 4. in accordance with the experiments described in Example 4, illustrates the results of a human IFNγ ELISA of cell culture supernatants of T cells positive for the 10D11 CAR of SEQ ID NO:28 and of T cells positive for the Clone2 CAR cultivated in NY-BR-1 and NY-BR-1.1 coated plates for 24 h. Experiments were performed in triplicates, error bars show SEM.

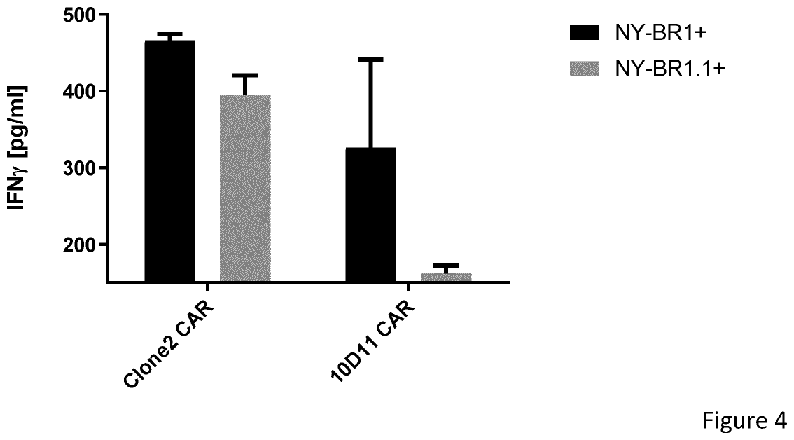

FIG. 5. in accordance with the experiments described in Example 5, illustrates the results of a degranulation assay of CAR-expressing Jurkat cells. Jurkat cells stably expressing the 10D11 CAR (SEQ ID NO:28) or the corresponding Clone2-derived CAR were co-cultivated with NY-BR-1 and NY-BR-1.1 transfected Bosc23 cells at a ratio of 1:1 for 24 h. Expression of the degranulation marker CD107a was determined by intracellular flow cytometric analysis. Mean Fluorescence Intensities are given.

FIG. 6. in accordance with the experiments described in Example 6, illustrates the results of a xCELLigence based killing assay of NY-BR-1 expressing pleural effusion cells by NY-BR-1 specific CAR+ T cells in an allogeneic setting. Primary T cells were isolated from a healthy donor, electroporated with DNA plasmid vectors encoding the 10D11 CAR expressing cassette (SEQ ID NO: 29) or the corresponding Clone2-derived CAR expressing cassette and co-cultivated with primary pleural effusion cells at a ratio of 1:1. Untransfected (mock) T cells served as controls. Target cell viability was monitored in real-time by measurement of the cell impedance (xCELLigence, ACEA Biosystems Inc.). Experiments were performed in triplicates, error bars show SEM.

FIG. 7. in accordance with the experiments described in Example 6, illustrates the results of a human IFNγ ELISA of NY-BR-1 specific CAR⁺ T cells co-cultivated with allogeneic pleural effusion cells. Endpoint analysis of the cell culture supernatants of the xCELLigence killing assay shown in FIG. 5 using an IFNγ ELISA detection kit. Experiments were performed in triplicates (mean values±s.e.m.; *, p<0.05; paired one-tailed Student's t-test).

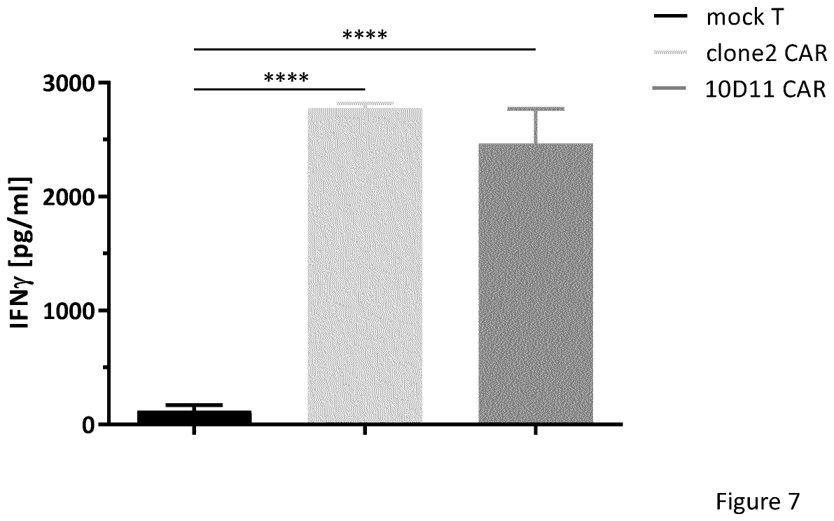

FIG. 8. in accordance with the experiments described in Example 7, illustrates the anti-tumour efficiency of murine CAR⁺ T cells in an allograft mouse model. NOD.CB17-Prkdc^{scid} mice engrafted with NY-BR-1⁺ Bosc23 derived subcutaneous tumours were treated with untransfected (mock) T cells or CAR⁺ T cells. Each group, except for control (n=9), consisted of seven mice. Tumour sizes were measured every three to four days. The average tumour volumes are displayed (mean values±s.e.m.; *, p<0.05; ***, p<0.001; Two-way ANOVA with Bonferroni post test for comparison with mock group). The corresponding survival rates are illustrated as Kaplan-Meier survival curves. Survival rates were compared with control group by using Log-rank (Mantel-Cox) test (*, p<0.05; **, p<0.01; ***≤0.001).

FIG. 9. in accordance with the experiments described in Example 8, illustrates the anti-tumour efficiency of human CAR⁺ T cells in a xenograft mouse model. NSG mice engrafted with NY-BR-1⁺ EO771 derived subcutaneous tumours were treated with untransfected (mock) T cells or CAR⁺ T cells. Each group consisted of seven mice. Tumour sizes were measured every three to four days. The average tumour volumes are given (mean values±s.e.m.; *, p<0.05; **, p<0.01; Two-way ANOVA with Bonferroni post test for comparison with mock group). The corresponding survival rates are illustrated as Kaplan-Meier survival curves.

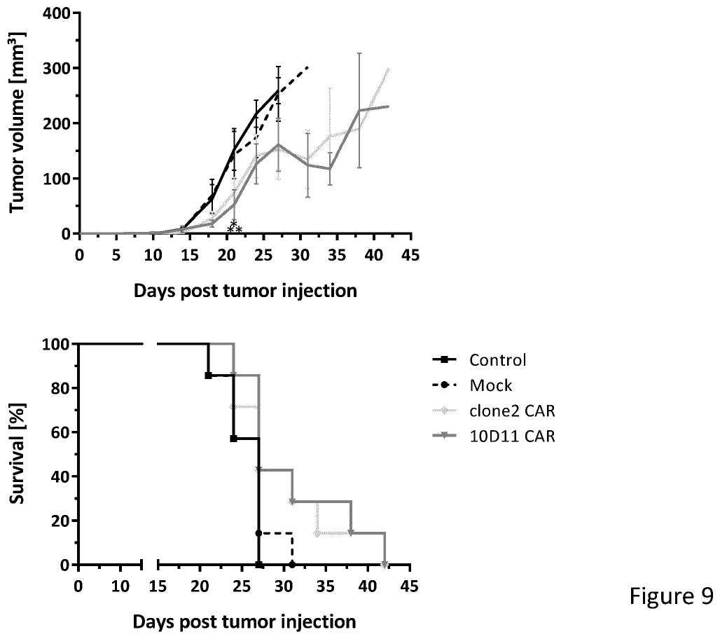

### DETAILED DESCRIPTION OF THE INVENTION

In order to provide a clear and consistent understanding of the specification and claims, and the scope to be given such terms, the following definitions are provided.

In the context of the present application, the term “immunoreactive molecule”—refers to a polypeptide molecule that specifically recognises and binds a target sequence or epitope of an antigen, i.e. a molecule that reacts with a target sequence or epitope of an antigen by specifically recognising and binding to this target sequence or epitope relying on the immunoglobulin (Ig) concept of target/epitope recognition known from molecules of the conventional Ig format (i.e. IgG, IgD, IgE, IgA and/or IgM). Accordingly, in particular embodiments, an immunoreactive molecule may comprise, at least, complementarity-determining regions (CDRs) of an Ig sufficient to confer target/epitope specificity and, in some instances, the immunoreactive molecule may comprise both variable heavy (V_{H}) and variable light (V_{L}) chain sequences of an Ig to confer target/epitope specificity. In some embodiments, an immunoreactive molecule may be an Ig as such, e.g. an IgG.

This, however, does not mean that the immunoreactive molecule of the present invention must still be in this Ig format. Instead, the immunoreactive molecule can be an Ig fragment or derivative thereof, e.g. a single-chain variable fragment (scFv) or an antigen-binding fragment (Fab—obtained through papain cleavage of an IgG; F(ab′)-obtained through pepsin cleavage of an IgG; and/or F(ab′)2-obtained pepsin cleavage of an IgG and subsequent β-mercaptoethanol treatment; etc.). Likewise, immunoreactive molecules of the present invention can be a new antibody format, for example and without limitation, a bi- or tri-specific antibody construct, a Diabody, a Camelid Antibody, a Domain Antibody, a Nanobody, a bivalent homodimer with two chains consisting of scFvs, a shark antibody, an antibody consisting of new-world primate framework plus non-new world primate CDR, a dimerised construct comprising CH₃+V_{L}+V_{H}, or an antibody conjugate (e.g. and antibody or fragment or derivative thereof linked to a toxin, a cytokine, a radioisotope or a label).

A further format of the immunoreactive molecules of the present invention is the chimeric antigen receptor (CAR) format. In this format, the immunoreactive molecule of the invention is a fusion polypeptide comprising an antibody-derived antigen recognition domain and, at least, a T-cell activation domain. Typically, in this format the chimeric antigen receptor also comprises a trans-membrane domain such that, when arranged on the surface of a transduced T-cell, the antigen recognition domain of the CAR is positioned in the extracellular space, while its T-cell activation domain is positioned intracellularly.

In the context of the present specification, the immunoreactive molecules are all polypeptide molecules that comprise antigen binding sequences such that the immunoreactive molecule, at least, recognises and binds to an epitope of NY-BR-1 comprising the antigen peptide sequence RGQVRKLEKMTKR (SEQ ID NO:1).

The skilled person knows how to translate a given antibody, which is still in the conventional Ig format, and which has proven to have specificity for a given target, such as the anti-NY-BR-1 10D11 antibody of SEQ ID NO:34 which specifically recognises and binds to human Ankyrin Repeat Domain-Containing 30A (NY-BR-1; UniProtKB Q9BXX3; SEQ ID NO:30), into a fragment or derivative thereof (such as into those described above) or into a new antibody or chimeric antigen receptor format (such as into those also described above) to arrive at the immunoreactive molecules of the present invention.

Specifically, the immunoreactive molecules of the present invention are polypeptide molecules comprising immunoglobulin antigen binding sequences directed at an epitope of human NY-BR-1 such that the immunoreactive molecule specifically recognises and binds to human NY-BR-1.

In this context, the phrase “specifically recognises and binds to” means that the immunoreactive molecule does not display cross-reactivity with epitopes of other antigens.

In particular, the immunoreactive molecules of the present invention, which specifically recognise and bind to human NY-BR-1 do not show cross-reactivity with human isoforms of NY-BR-1 such as human Ankyrin Repeat Domain-Containing Protein 30B (NY-BR-1.1; UniProtKB Q9BXX2; SEQ ID NO:31) or Isoform 2 thereof (NY-BR-1.1 Isoform 2; UniProtKB Q9BXX2-2; SEQ ID NO:32). This requires that the epitope, against which the immunoreactive molecule is directed, is unique in sequence and/or conformation. Preferably, the immunoreactive molecule specifically recognises and binds to an epitope of NY-BR-1 comprising the antigen peptide sequence RGQVRKLEKMTKR (SEQ ID NO:1).

Unless specifically indicated otherwise, in the context of the present specification, immunoreactive molecules, in particular the immunoreactive molecules of the present invention, that specifically recognise and bind to NY-BR-1 may be comprised in larger structures, e.g. may be covalently or non-covalently linked to carrier molecules, retardants, and other excipients. For example, the immunoreactive molecules of the present invention may be linked to a chromatography material/resin useful in affinity chromatography methods for the purification of human NY-BR-1. Alternatively, or additionally, the immunoreactive molecule may a polypeptide as specified above, which is comprised in a fusion polypeptide with one or more other peptides, individually or in combination serving e.g. as a tag for detection and/or purification, a linker or spacer, a trans-membrane domain, an intracytoplasmic domain, a signal and/or transport sequence or to extend the in vivo half-life of the immunoreactive molecule.

The term “detectable tag” refers to a stretch of amino acids added to or introduced into such a fusion polypeptide. Preferably, the detectable tag is added to the C- or N-terminus of the immunoreactive molecule or a fusion polypeptide comprising the same. This stretch of amino acids preferably allows for the detection of the immunoreactive molecule or fusion polypeptide comprising the same by an antibody, which specifically recognises and binds to the tag, or it preferably allows for visualisation, e.g. in case of fluorescent tags. Detectable tags are particularly useful if the immunoreactive molecules of the present invention are utilised in Companion Diagnostics (CDx) as the detection, identification and/or visualisation is particularly important. Preferred tags are the Myc-tag, FLAG-tag, His-tag, HA-tag, GST-tag, Strep-tag or a fluorescent protein tag, e.g. EGFP- or EYFP-tag. These tags are well known in the art and are routinely used by the skilled person. Other fusion polypeptides may comprise the immunoreactive molecule fused to or fused with amino acids or other modifications, which serve as mediators of secretion, mediators of blood-brain-barrier passage, as cell-penetrating peptides and/or immune stimulants.

“human Ankyrin Repeat Domain-Containing Protein 30A (NY-BR-1)”—means the human protein of SEQ ID NO:30 assigned the identifier “UniProtKB Q9BXX3” in the freely accessible database UniProt available at www.uniprot.org.

“cross-reactivity”—means that an immunoreactive molecule targeted against one specific antigen recognizes at least a second antigen that has similar structural regions such as, e.g., isoforms of the targeted antigen or proteins sharing a similar domain structure with targeted antigen. As such, the phrase “shows no cross-reactivity” means that an immunoreactive molecule does not recognize any other antigens. Typically, this is only achievable, if the immunoreactive molecule is specific to a unique epitope of the targeted antigen.

“human Ankyrin Repeat Domain-Containing Protein 30B (NY-BR-1.1)”—means the human protein of SEQ ID NO:31 assigned the identifier “UniProtKB Q9BXX2” in the freely accessible database UniProt available at www.uniprot.org.

“human Ankyrin Repeat Domain-Containing Protein 30B Isoform 2 (NY-BR-1.1 Isoform 2)”—means the human protein of SEQ ID NO:32 assigned the identifier “UniProtKB Q9BXX2-2” in the freely accessible database UniProt available at www.uniprot.org.

The phrase “a cancer characterised by the expression of the cancer associated antigen NY-BR-1” means a malignant disease characterised by the development of abnormal cells that divide uncontrollably and have the ability to infiltrate and destroy normal body tissue (cancer), wherein the majority of those cancer cells express the human Ankyrin Repeat Domain-Containing Protein 30A (NY-BR-1). Such expression may, e.g., be proven by way of performing reverse transcriptase-polymerase chain reaction analysis of the cancer cells or by detection using immunoreactive molecules, which specifically recognise NY-BR-1.

The term “companion diagnostics (CDx) method” means a method suitable to provide information that is essential for the safe and effective use of a corresponding drug or biological therapeutic product.

CDx methods help a health care professional to determine whether the particular benefits of a corresponding drug or therapeutic product to patients will outweigh any potential serious side effects or risks. As such, a CDx method can:
identify patients who are most likely to benefit from a particular drug or therapeutic product;
identify patients likely to be at increased risk for serious side effects as a result of treatment with a particular drug or therapeutic product; or
monitor response to treatment with a particular drug or therapeutic product for the purpose of adjusting treatment to achieve improved safety or effectiveness.

In addition to the above definitions, and unless the context clearly requires otherwise, throughout the description and the claims, terms used herein are to be given their ordinary plain English meaning. As used herein the words “comprise”, “comprising”, “having”, “including” and the like are to be construed in an inclusive sense as opposed to an exclusive or exhaustive sense; that is to say, in the sense of “including, but not limited to”.

Further, reference throughout this specification the terms “preferably”, “more preferably”, “most preferably”, “particularly”, “more particularly”, “most particularly”, “specifically”, “more specifically”, “typically”, “usually”, “generally” or similar terms are used in conjunction with optional features without limitation as to other and/or further features. As such, features introduced by these or similar terms are not intended to restrict the scope of the claims. The invention may, as the person skilled in the art will recognise, be performed using alternative features.

Similarly, reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

As used herein, the term “exemplary” is used in the sense of providing examples, as opposed to indicating quality. That is, an “exemplary embodiment” is an embodiment provided as an example, as opposed to necessarily being an embodiment of exemplary quality.

The “degree of identity” (e.g. expressed as “% identity”) between two biological sequences, preferably DNA, RNA or amino acid sequences, can be determined by algorithms well known in the art. Preferably, the degree of identity is determined by comparing two optimally aligned sequences over a comparison window, where the fragment of sequence in the comparison window may comprise additions and/or deletions (e.g. gaps, insertions and/or overhangs) compared to the sequence it is compared to for optimal alignment. The percentage is calculated by: (a) determining, preferably over the whole length of the polynucleotide or polypeptide sequence, the number of positions at which the identical residue occurs in both sequences to yield the number of matched positions, (b) dividing the number of matched positions by the total number of positions in the comparison window, and (c) multiplying the result by 100 to arrive at the percentage of sequence identity. Optimal alignment of sequences for comparison may be conducted by accepted methods known to the person of skill in the art, such as by the local homology algorithm of Smith and Waterman (1981), by the homology alignment algorithm of Needleman and Wunsch (1970), by the search for similarity method of Pearson and Lipman (1988), by computerised implementations of these algorithms (e.g. GAP, BESTFIT, BLAST, PASTA, TFASTA, etc.) or by visual inspection. Given that two sequences have been identified for comparison, GAP and BESTFIT are preferably employed to determine their optimal alignment and, thereby, their degree of identity. Preferably, the default values of 5.00 for gap weight and 0.30 for gap weight length are used.

In the context of biological sequences referred to herein, the term “essentially identical” indicates a % identity of at least 80%, preferably at least 90%, more preferably at least 98%, most preferably 99%. As will be understood, the term “essentially identical” includes 100% sequence identity. The aforesaid applies to the term “essentially complementary” mutatis mutandis.

The term “fragment” of a biological macromolecule, preferably a polynucleotide or a polypeptide, as used throughout the specification, is to be understood in a broad sense, namely as relating to any sub-part, preferably sub-domain, of the respective macromolecule comprising the indicated sequence, structure and/or function. The term, therefore, includes sub-parts generated by actual fragmentation of a biological macromolecule, but also sub-parts derived from the respective macromolecule in an abstract manner, e.g. in silico. As such, fragments of an immunoreactive molecule such as an immunoglobulin, include Fc or Fab fragments as well as e.g. single-chain antibodies, bispecific antibodies and nanobodies.

### Immunoreactive Molecules of the Invention

As already indicated above, in a first aspect, the present invention relates to immunoreactive molecules, which specifically recognise and bind to human Ankyrin Repeat Domain-Containing Protein 30A (NY-BR-1; UniProtKB Q9BXX3; SEQ ID NO: 30). However, other than previously-known NY-BR-1 immunoreactive antibodies, the immunoreactive molecules of the present invention show no cross-reactivity to human Ankyrin Repeat Domain-Containing Protein 30B (NY-BR-1.1; UniProtKB Q9BXX2; SEQ ID NO:31) or Isoform 2 thereof (NY-BR-1.1 Isoform 2; UniProtKB Q9BXX2-2; SEQ ID NO:32). The high degree of target specificity as well as the lack of cross-reactivity makes the immunoreactive molecules of the present invention particularly suitable for the use in the treatment of NY-BR-1 positive cancers because undesirable targeting of healthy tissues expressing NY-BR-1.1 or its Isoform 2 is avoided.

The most promising of the previously-known NY-BR-1 antibody, namely “Clone2” (Seil et al. 2007), not only recognises and binds to NY-BR-1 but, undesirably, also recognises and binds to the closely related proteins NY-BR-1.1 and NY-BR-1.1 Isoform 2. Specifically, retrospective analysis of Clone2's epitope-specificity has revealed that Clone2 recognises and binds to an epitope of NY-BR-1 comprising the antigen peptide sequence of SEQ ID NO:33. However, a near-identical sequence (with only a single amino acid difference) is also present in NY-BR-1.1 and NY-BR-1.1 Isoform 2. In contrast, the immunoreactive molecules of the present invention specifically recognise and bind to an epitope of NY-BR-1 comprising the antigen peptide sequence of SEQ ID NO:1, of which neither an identical nor near-identical sequence is present in NY-BR-1.1 and NY-BR-1.1 Isoform 2. The 13 amino acid long antigen-binding sequence of SEQ ID NO:1 is located only at amino acid residues 48 to 60 within the 1397 amino acid long full-length sequence of NY-BR-1 (i.e. of SEQ ID NO: 30), i.e. fairly close to the amino (N) terminus of NY-BR-1 and still N-terminally of NY-BR-1's first of six Ankyrin repeat domains. In contrast, it has been found that Clone2 recognises the antigen peptide sequence of SEQ ID NO:33, which is located further towards the carboxy (C) terminus within SEQ ID NOs: 30, 31 and 32. While still being located relatively centrally within SEQ ID NOs: 30 and 32, it is located even closer to the C-terminus in SEQ ID NO:32. In each the case antigen peptide sequence of SEQ ID NO:33 is located C-terminal of the Ankyrin repeat domains within SEQ ID NOs: 30, 31 and 32.

In preferred embodiments, the immunoreactive molecule has an antigen binding region comprising a mouse variable light chain (V_{L}) sequence including complementarity-determining regions (CDRs) 1 to 3 of SEQ ID NOs: 2 to 4, respectively, and a mouse variable heavy chain (V_{H}) sequence including corresponding CDRs 1 to 3 of SEQ ID NOs: 5 to 7, respectively. For example, the V_{L} and V_{H} sequences are SEQ ID NO:8 (encoded by nucleotide sequence of SEQ ID NO: 9) and SEQ ID NO: 10 (encoded by nucleotide sequence of SEQ ID NO:11).

As shown in the Figures and Examples, immunoreactive molecules comprising these particular V_{L} and V_{H} sequences do not cross-react with either NY-BR-1.1 or NY-BR-1.1 Isoform 2.

| | Length | | |
|---|---|---|---|
| SEQ ID | (AAs or | | |
| NO: | DNA bps) | | Name/Domains |
| 2 | 7 | AAs | 10D11 variable light chain CDR1 |
| 3 | 4 | AAs | 10D11 variable light chain CDR2 |
| 4 | 7 | AAs | 10D11 variable light chain CDR3 |
| 5 | 8 | AAs | 10D11 variable heavy chain CDR1 |
| 6 | 10 | AAs | 10D11 variable heavy chain CDR2 |
| 7 | 9 | AAs | 10D11 variable heavy chain CDR3 |
| 8 | 107 | AAs | 10D11 variable light chain |
| 9 | 321 | bps | 10D11 variable light chain coding sequence |
| 10 | 121 | AAs | 10D11 variable heavy chain |
| 11 | 363 | bps | 10D11 variable heavy chain coding sequence |

As explained above, even when comprising mouse V_{L} and V_{H} sequences, the immunoreactive molecule is not limited to mouse antibodies as such, but—while it may be a monoclonal mouse antibody—it may also be a single-chain variable fragment-fragment crystallisable region (scFv-Fc) fusion protein or a chimeric antigen receptor (CAR) comprising such mouse V_{L} and V_{H} sequences.

In particular embodiments, the immunoreactive molecule may nevertheless be a full-length monoclonal mouse IgG, preferably a full-length monoclonal mouse IgG2a, wherein said full-length monoclonal mouse IgG2a preferably comprises SEQ ID NO: 12 (encoded by nucleotide sequence of SEQ ID NO:13) and SEQ ID NO: 14 (encoded by nucleotide sequence of SEQ ID NO:15). In some embodiments, the immunoreactive molecule may be the full-length monoclonal mouse IgG of SEQ ID NO: 34.

| | Length | | |
|---|---|---|---|
| SEQ ID | (AAs or | | |
| NO: | DNA bps) | | Name/Domains |
| 12 | 233 | AAs | 10D11 IgG2a isotype variable light chain |
| 13 | 699 | bps | 10D11 IgG2a isotype variable light chain |
| | | | coding sequence |
| 14 | 470 | AAs | 10D11 IgG2a isotype variable heavy chain |
| 15 | 1407 | bps | 10D11 IgG2a isotype variable heavy chain |
| | | | coding sequence |
| 34 | 703 | AAs | 10D11 antibody |

Alternatively, the immunoreactive molecule may be a single-chain variable fragment-fragment crystallisable region (scFv-Fc) fusion protein comprising either a mouse or a human IgG1 Fc domain, preferably comprising either SEQ ID NO:16 (encoded by nucleotide sequence of SEQ ID NO:17) or SEQ ID NO:18 (encoded by nucleotide sequence of SEQ ID NO: 19).

| | Length | | |
|---|---|---|---|
| SEQ ID | (AAs or | | |
| NO: | DNA bps) | | Name/Domains |
| 16 | 501 | AAs | 10D11 scFv-mouse IgG1-Fc |
| 17 | 1503 | bps | 10D11 scFv-mouse IgG1-Fc coding sequence |
| 18 | 504 | AAs | 10D11 scFv-human IgG1-Fc |
| 19 | 1497 | bps | 10D11 scFv-human IgG1-Fc coding sequence |

In embodiments where the immunoreactive molecule is a chimeric antigen receptor (CAR), the CAR has an antigen recognition domain, which comprises SEQ ID NO:20 (encoded by nucleotide sequence of SEQ ID NO:21). SEQ ID NO:20 itself comprises the variable light chain and variable heavy chain sequences of SEQ ID NOs: 8 and 10, respectively. Further, the CAR may also comprise a CD28-derived sequence in its T-cell activation domain, preferably SEQ ID NO:22 (encoded by nucleotide sequence of SEQ ID NO:23). Alternatively or additionally, the CAR may also comprise a CD3zeta-derived sequence in its transmembrane domain, preferably SEQ ID NO:24 (encoded by nucleotide sequence of SEQ ID NO:25). Still further—or again alternatively—the CAR may comprise an OX 40-derived sequence in its T-cell activation domain, preferably SEQ ID NO:26 (encoded by nucleotide sequence of SEQ ID NO:27).

| | Length | | |
|---|---|---|---|
| SEQ ID | (AAs or | | |
| NO: | DNA bps) | | Name/Domains |
| 20 | 477 | AAs | 10D11 CAR antigen recognition |
| 21 | 1431 | bps | 10D11 CAR antigen recognition coding sequence |
| 22 | 68 | AAs | CD28-derived amino acid sequence |
| 23 | 204 | bps | CD28-derived nucleotide sequence |
| 24 | 113 | AAs | CD3zeta-derived amino acid sequence |
| 25 | 339 | bps | CD3zeta-derived nucleotide sequence |
| 26 | 36 | AAs | OX40-derived amino acid sequence |
| 27 | 108 | bps | OX40-derived nucleotide sequence |

In embodiments where the immunoreactive molecule of the invention is a CAR, it may comprise all of the preceding elements, in conjunction with short amino acid sequences, serving as leader-, linker, or hinge sequences between the individual elements of the immunoreactive molecule. In particular, the CAR may comprise or essentially consist of SEQ ID NO. 28 (encoded by nucleotide sequence of SEQ ID NO: 29).

| | | |
|---|---|---|
| | Length | |
| SEQ ID | (AAs or | |
| NO: | DNA bps) | Name/Domains |

| | | | |
|---|---|---|---|
| 28 | 694 | AAs | 10D11 CAR full amino acid sequence |
| 29 | 2082 | bps | 10D11 CAR full coding sequence |

As set out above, the immunoreactive molecules of the present invention provide another tool for physicians combatting cancer. As such, it will be understood that the immunoreactive molecules of the invention are for use in the treatment of cancer. Accordingly, the present invention also encompasses methods of treating cancer, wherein the method comprises administering the immunoreactive molecules according to the first aspect as disclosed herein to a subject in need thereof. Similarly, use of an immunoreactive molecule according to the first aspect as disclosed herein in the manufacture of a medicament for the treatment of cancer constitutes part of the present invention.

As will be understood, the immunoreactive molecules and methods of the present invention are particularly suited for targeting cancer cells, which express the cancer associated antigen NY-BR-1. Due to the established expression patterns of NY-BR-1 in solid tumours, but without limitation, the cancer is typically testicular cancer or breast cancer.

Further, the present invention relates to use of the immunoreactive molecule according to the first aspect as disclosed herein in a method of detecting NY-BR-1 in a sample, wherein, optionally, the sample is a sample previously obtained and/or derived from a cancer patient and the method is a companion diagnostics (CDx) method.

Typically, the method by which NY-BR-1 may be detected in a sample, is selected from the group consisting of: immunohistochemical staining methods; membrane-mediated protein blotting methods; and flow cytometry-based methods.

The sample is generally a tissue sample, a primary cell sample or a cell line sample. For example, at different stages during the course of an anti-cancer treatment regime, tissue samples from a patient suffering from a NY-BR-1 positive cancer may be taken to assess whether the chosen anti-cancer treatment regime is effective or not for treating the particular patient's cancer. Specifically, the immunoreactive molecules of the present invention may be used in the aforementioned methods to detect and/or quantify whether a relative reduction of NY-BR-1 positive cells within the patient's samples can be achieved.

In the event that the chosen anti-cancer treatment regime is effective, a decline in NY-BR-1 positive cells may be observed compared to a previous sample taken earlier during the course of treatment. Conversely, if the amount of NY-BR-1 positive cells remains unchanged or only declines very slowly in consecutively taken patient samples, the physician may adjust the treatment regime in order to achieve more effective treatment for the specific patient.

Alternatively or additionally, the immunoreactive molecules of the present invention may be used to isolate NY-BR-1 positive cells from a patient's tissue sample for further characterisation. For example, a culture of primary cells from the tissue sample (either isolated NY-BR-1 positive cells only or a culture representative of the different cell types within the sample) can be established such as to assess the patient's cells' specific sensitivities to the chemotherapeutic agents available for the treatment of the patient.

In this context, the methods of the present invention may also be used to detect the efficacy of a chosen anti-cancer treatment regime on a previously established (i.e. non-primary) cell line most representative of the tumour type of the patient to be treated such as to gauge the likelihood of efficacy of the chosen anti-cancer treatment regime. The skilled person will understand that an anti-cancer treatment regime comprises several variables, which may ultimately be adjusted based on the results obtained in CDx methods. In particular, the physician may consider adjusting or altering the dosage of a particular anti-cancer agent, the combination and relative amounts of anti-cancer agents, the administration conditions, the intervals between administrations etc. in response to the assessment of efficacy obtained through the use of the immunoreactive molecule of the first aspect as disclosed herein in such methods of detecting NY-BR-1 in a sample.

### EXAMPLES

The invention is further described by the following non-limiting Examples.

### Example 1: Detection of NY-BR-1 and NY-BR-1.1 Transfected Bosc23 Cells by Full-Length Murine Antibodies 10D11 or Clone2

In order to evaluate the specificity and binding capacity of the 10D11 antibody to the NY-BR-1 protein as well as possible cross-reactivities with the homologue NY-BR-1.1, Bosc23 cells were transiently transfected with the full-length proteins NY-BR-1 and NY-BR-1.1 and subsequently examined by FACS with the full-length murine 10D11 antibody. The results were compared with the binding and cross-reactivity properties of the full-length murine Clone2 antibody, which has a known specificity for NY-BR-1.

Briefly, Bosc23 cells were transfected using 4 μg DNA in 600 μl Opti-MEM and 12 μl Lipofectamine (Lipofectamine 2000, ThermoFisher) per well (6 well plate). 24 h post transfection, cells were stained using increasing concentrations of the murine full-length 10D11 (2.5 μg, 5 μg, 10 μg, 25 μg, 50 μg/mL) or Clone2 antibodies (5 μg per). Following 1 h incubation at 4° C., cells were washed twice with FACS buffer and incubated with the secondary PE-conjugated anti-mouse Fcγ subclass I specific antibody (Jackson Immuno Research) for 30 minutes at 4° C. After two washing steps with FACS buffer, flow cytometric analyses were carried out on a FACS Canto II device (BD Biosciences).

Compared to an isotype control, and despite increasing antibody concentrations, the flow cytometric analyses revealed no binding of the purified 10D11 antibody to NY-BR-1.1 (no significant shift in the Mean Fluorescent Intensity-MFI; NY-BR-1.1: isotype control MFI=47; 10D11 MFI at 2.5 μg/mL=49, 10D11 MFI at 5 μg/mL=50, 10D11 MFI at 10 μg/mL=50, 10D11 MFI at 25 μg/mL=54, 10D11 MFI at 50 μg/mL=56), whereas a dose-dependent sensitivity to NY-BR-1 was observed (NY-BR-1: isotype control MFI=48; 10D11 MFI at 2.5 μg/mL=135, 10D11 MFI at 5 μg/mL=151, 10D11 MFI at 10 μg/mL=158, 10D11 MFI at 25 μg/mL=178, 10D11 MFI at 50 μg/mL=181).

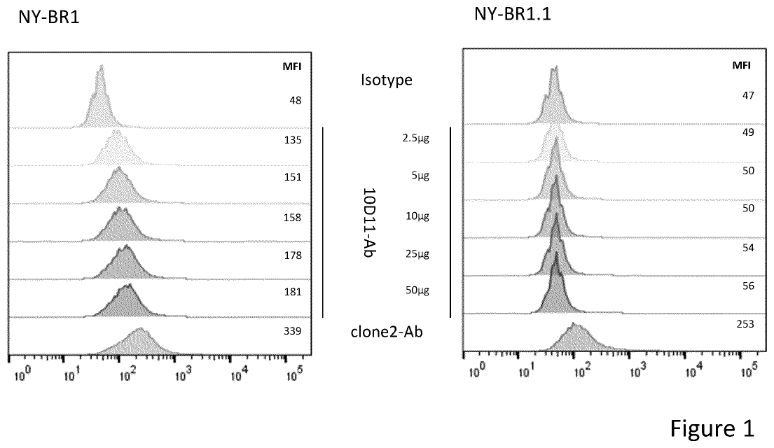

In contrast, the purified Clone2 antibody clearly binds to both NY-BR-1 and NY-BR-1.1 (NY-BR-1: Clone2 MFI at 5 μg/mL=339; NY-BR-1.1: Clone2 MFI at 5 μg/mL=253). Results are shown in FIG. 1.

### Example 2: Epitope Discovery of 10D11

In order to determine the NY-BR-1 epitope that is specifically recognised and bound by the full-length murine 10D11 antibody of SEQ ID NO:34, the proprietary technology PEPperMAP® by PEPperPRINT GmbH, Heidelberg was used.

Briefly, peptides of the amino acids 1-367 of NY-BR-1 (i.e. of SEQ ID 30) were spotted onto a microarray chip. The C- and N-termini of the antigen were elongated by neutral GS linkers to avoid truncated peptides. The protein sequence was then translated into 13mer peptides with a peptide-peptide overlap of 12 amino acids. After peptide synthesis, all peptides were cyclized via a thioether linkage between a C-terminal cysteine side chain thiol group and an appropriately modified N-terminus. The resulting conformational NY-BR-1 peptide microarrays contained 1,116 different cyclic constrained peptides printed in duplicate (2,232 peptide spots), and were framed by additional HA (YPYDVPDYAG, 104 spots) control peptides. After 10 minutes pre-swelling in standard buffer and 60 minutes in blocking buffer, the peptide arrays were initially incubated with the secondary antibodies at a dilution of 1:5000 for 60 minutes at room temperature to avoid background interactions with the antigen-derived peptides. After 2×1 minute washing and 30 minutes swelling in standard buffer, the Type 1 Mapping arrays were incubated overnight at 4° C. with 10D11 monoclonal antibody at a concentration of 100 μg/ml. Repeated washing in standard buffer (2×1 minute) was followed by incubation with the secondary antibody for 30 minutes at room temperature and a dilution of 1:5000. After 2×1 minute washing in standard buffer, the microarray was rinsed with Millipore® water and dried in a stream of air. Read-out with an Odyssey Imaging System was done at a resolution of 21 μm and a scanning intensity of 7.

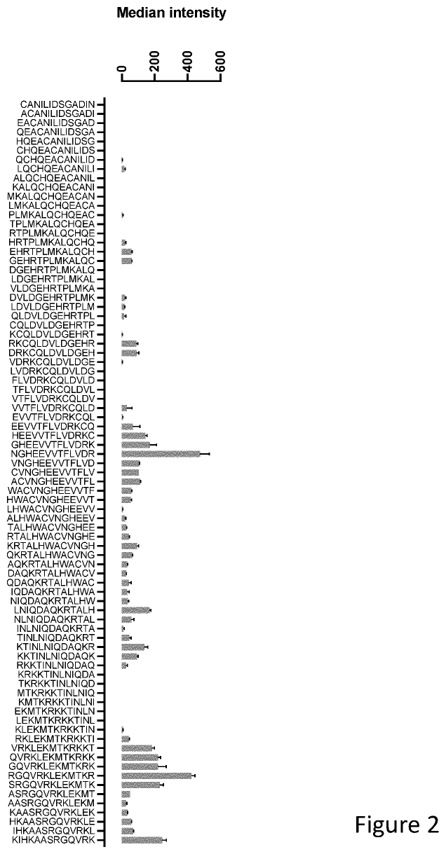

Median intensities of read-out were plotted and revealed a cluster of 5 overlapping peptides showing the highest intensity. Results are shown in FIG. 2. The consensus sequence of these peptides is identified as the NY-BR-1 epitope specifically recognised and bound by the 10D11 antibody of SEQ ID NO:1.

### Example 3: Detection of NY-BR-1 and NY-BR-1.1 Transfected Bosc23 Cells by 10D11 or Clone2 Derived scFv-Fc Fusion Proteins

Next to the full-length murine 10D11 antibody of SEQ ID NO:34, the corresponding fusion protein consisting of the 10D11scFv and human Fc domain (SEQ ID NO:18) were also studied for their binding and cross-reactivity properties against the full-length proteins NY-BR-1 and NY-BR-1.1. The results were compared with the binding capacities of the Clone2 derived fusion protein (Clone2scFv-Fc). Therefore, NY-BR-1 and NY-BR-1.1 transiently transfected Bosc23 cells were generated and analysed by flow cytometry, as described in Example 1. Secondary PE-conjugated anti-human Fcγ fragment specific (Jackson Immuno Research) antibody was used to detect binding of the 10D11scFv-hFc (SEQ ID NO:18) and the Clone2scFv-hFc fusion proteins.

Compared to an isotype control, all fusion constructs exhibit strong binding capacities to NY-BR-1 (NY-BR-1: isotype control MFI=58; 10D11scFv-hFc MFI=1037; Clone2scFv-hFcMFI=466), additionally, the Clone2 derived fusion protein also shows a clearly detectable binding to the NY-BR-1.1 protein (NY-BR-1.1: isotype control MFI=60; Clone2scFv-hFc MFI=489). In contrast, the 10D11 derived fusion protein does not bind to NY-BR-1.1 (NY-BR-1.1: isotype control MFI=60; 10D11scFv-hFc MFI=134), which is consistent with the results obtained with the full-length 10D11 antibody in Example 1. Results are shown in FIG. 2.

### Example 4: Activation Analysis of 10D11 CAR Expressing T Cells after Co-Cultivation with NY-BR-1 and NY-BR-1.1 Full-Length Peptides

To investigate the effects of possible cross-reactivities in a CAR format, primary T cells were isolated from a healthy donor and transduced with lentiviral vectors encoding the 10D11 CAR gene expression cassette comprising SEQ ID NO:29 or a corresponding Clone2 CAR gene expression cassette. For the isolation of primary T cells, blood samples were taken in EDTA collection tubes and thoroughly pipetted onto a layer of Ficoll (Ficoll Paque, GE Healthcare; density: 1.077) in a 50 ml Falcon™ tube. Following centrifugation at 2200 rpm for 20 minutes without break, the formed ring of lymphocytes was transferred into a new 50 mL Falcon™ tube and washed twice with 20 mL PBS (centrifugation at 1500 rpm for 10 minutes). Afterwards, cell numbers were determined for the subsequent isolation of human T cells with the Pan T cell Isolation Kit (Miltenyi) according to the manufacturer's instructions. Following 48 h of cultivation in TexMACS GMP medium (Miltenyi), supplemented with T cell TransAct™ (1:100, Miltenyi), IL-7 (5 ng/ml) and IL-15 (5 ng/ml), T cells were lentivirally transduced with the 10D11 or Clone2 CAR cassettes under the addition of Polybrene (8 μg/mL) at an MOI of 5 and cultivated in TexMACS GMP medium (Miltenyi), supplemented with IL-7 (5 ng/ml) and IL-15 (5 ng/mL). 48h post transduction, the expression levels of the 10D11 CAR of SEQ ID NO:28 and of the corresponding Clone2 CAR were determined by flow cytometric analyses. Here, T cells were stained with the APC-conjugated anti-human CD3 (BD Biosciences, clone: UCHT1) and PE-conjugated anti-human Fcγ fragment specific (Jackson Immuno Research) antibodies for 30 minutes at 4° C., followed by two washing steps with FACS buffer (centrifugation at 1500 rpm for 5 minutes) and final analysis on the FACS Canto II device (BD Biosciences). For subsequent co-cultivation with the NY-BR-1 and NY-BR-1.1 proteins, 96 well plates were coated with glutathione casein (100 ng/well) overnight at 4° C. followed by coating with GST-tagged NY-BR-1 and NY-BR-1.1 protein containing cell lysates, which derived from transfected HEK 293T cells. Clone2 or 10D11 CAR expressing T cells as well as non-transduced (mock) T cells were cultivated in the NY-BR-1/NY-BR-1.1 coated 96 well plates (5×10⁵ CAR⁺ or mock T cells in 200 μl TexMACS GMP medium+IL-7/IL-15 per well) for 24 h. The level of CAR⁺ T cell activation was determined through the concentration of IFNγ in the cell culture supernatants using the BD OptEIA Human IFNγ ELISA set (BD Biosciences) according to the manufacturer's instructions.

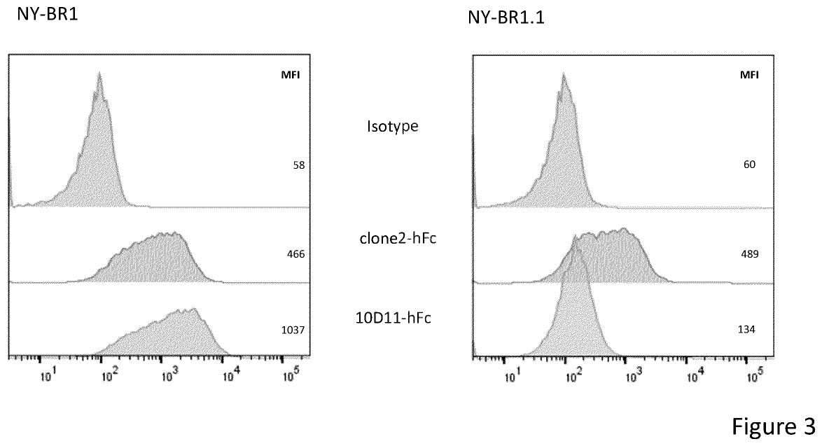

10D11 CAR⁺ T cells secreted increased amounts of IFNγ when cultivated in NY-BR-1 but not in NY-BR-1.1 coated plates, whereas Clone2 CAR⁺ T cells produced IFNγ in both coating variants (see FIG. 3). These results confirm that there are no cross-reactivities of all tested 10D11 formats (antibody, fusion protein, CAR) with the NY-BR-1.1 protein. Furthermore, these examples prove the specificity of the 10D11 CAR of SEQ ID NO:28 for the NY-BR-1 protein.

### Example 5: Activation Analysis of 10D11 CAR Expressing Jurkat Cells after Co-Cultivation with NY-BR-1 and NY-BR-1.1 Expressing Bosc23 Cells

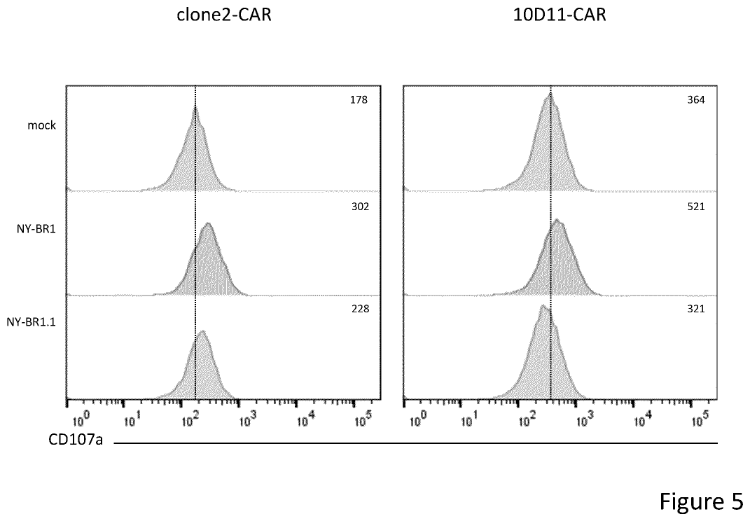

To reproduce the observed effects of isolated NY-BR-1 and NY-BR-1.1 proteins to the membrane bound target variants, Bosc23 cells were transfected with the NY-BR-1 and NY-BR-1.1 proteins, as described in Example 1, and—additionally—stable 10D11 or Clone2 CAR-expressing Jurkat cell lines were generated by lentiviral transduction. For this purpose, 1×10⁵ Jurkat cells were cultivated in 1 mL RPMI medium (containing 10% FCS) in 24 well plates and lentivirally transduced with vector including the 10D11 CAR expression cassette comprising SEQ ID NO:29 or with a vector including a corresponding Clone2 CAR gene expression cassette together with the puromycin resistance gene at an MOI of 10. The medium was replaced with fresh DMEM 24 h post transduction. After additional two days, transduced Jurkat cells were washed, transferred to a 96 well plate format and cultivated in DMEM medium, supplemented with puromycin (final concentration: 2 μg/mL), in order to select CAR⁺ Jurkat cells over a period of two to three weeks. The CAR expression levels of the resulting clones were determined using flow cytometric analyses. Cells were stained with the APC-conjugated anti-human Fcγ fragment specific antibody (Jackson Immuno Research) for 30 minutes at 4° C. Following two washing steps with FACS buffer (centrifugation at 1500 rpm for 5 minutes), cells were analysed on a FACS Canto II device (BD Biosciences). Thereafter, effector cells (Clone2 or 10D11 CAR⁺ Jurkat) were co-incubated with target cells (NY-BR-1, NY-BR-1.1 transfected Bosc23) at a ratio of 1:1 in a 96 well format for 24 h. The activation levels of the CAR expressing Jurkat cells were determined using a CD107a degranulation assay. For this, cells were fixed with 200 μL of Cytofix/Cytoperm solution (BD Biosciences) for 15 minutes at 4° C. Thereafter, cells were washed twice with 400 μL of Perm/Wash solution (BD Biosciences) and stained with the PE-Cy7-conjugated anti-human CD107a antibody (BD Biosciences clone H4A3, 5 μl in Perm/Wash solution) for 30 minutes at 4° C. Following two additional washing steps with Perm/Wash solution, cells were resuspended in FACS buffer and analysed on a FACS Canto II device (BD Biosciences). The given Mean Fluorescence Intensities (MFIs) in FIG. 5 demonstrated that the Jurkat cells expressing the 10D11 CAR of SEQ ID NO:28 are specifically activated through membrane bound NY-BR-1 but not NY-BR-1.1 proteins. As expected, Clone2 CAR⁺ Jurkat cells were activated by both NY-BR-1 and NY-BR-1.1 transfected Bosc23 cells, confirming the previous results.

### Example 6: NY-BR-1 Expressing Pleural Effusion Cells are Eliminated by 10D11 CAR⁺ T Cells in an Allogeneic Setting

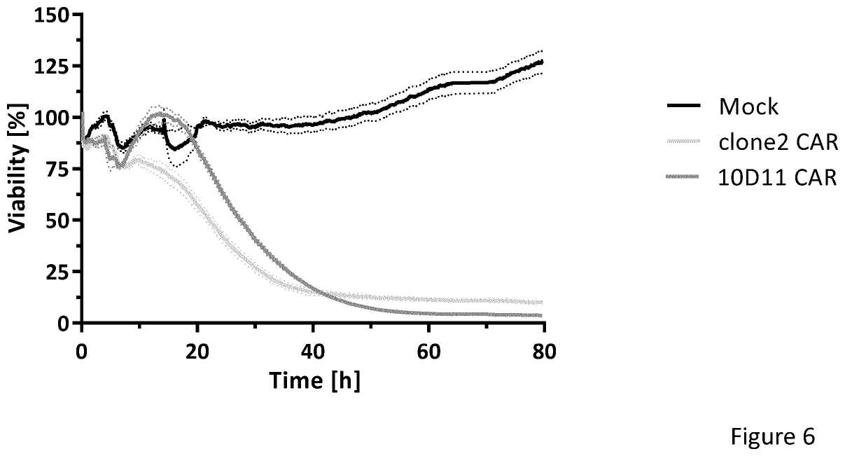

The functionality of T cells expressing the 10D11 CAR of SEQ ID NO:28 was assessed in a real-time killing assays of primary pleural effusion cells derived from a breast cancer patient. First, primary pleural effusion cells HD-A-213 were analysed for NY-BR-1 expression via flow cytometry with the primary Clone2 (3 μg/mL) and secondary APC-conjugated anti-mouse Fcγ subclass I specific antibodies (Jackson Immuno Research), as described in Example 1, and exhibited target expression levels of around 15 to 20%. In addition, primary T cells were isolated from a healthy donor, as described in Example 4, and electroporated with DNA plasmid vectors encoding the 10D11 or Clone2 CAR expression cassettes together with S/MAR based motifs for episomal maintenance. Electroporation was performed using the Neon Transfection System [ThermoFisher (5×10⁶ T cells and 10 μg DNA per shock)]. Electroporated T cells were further cultivated in TexMACS GMP medium (Miltenyi) supplemented with IL-7 (5 ng/ml) and IL-15 (5 ng/ml). 24 h post transfection, CAR expression levels of the electroporated T cells were determined by flow cytometry using the APC-conjugated anti-human CD3 (BD Biosciences, clone: UCHT1) and PE-conjugated anti-human Fcγ fragment specific (Jackson Immuno Research) antibodies, as described in Example 4. In order to analyse the lytic capacity of the 10D11 or Clone2 CAR⁺ T cells (effector cells), pleural effusion cells (target cells) were seeded in an E-Plate 96 (30000 cells/well, triplicate). Real-time monitoring of the cell impedance was performed by the xCELLigence RTCA instrument (ACEA Biosciences Inc.). Once target cells were adherent, effector cells were added at an effector to target ratio of 1:1. Untransfected (mock) T cells served as controls. Real-time measurement of cell impedance continued every 5 minutes for another 80 h. At the end of the experiment, the level of T cell activation was determined through detection of IFNγ in the cell culture supernatants using the BD OptEIA Human IFNγ ELISA set (BD Biosciences) according to the manufacturer's instructions. It was shown that T cells positive for the 10D11 CAR of SEQ ID NO:28 specifically lyse NY-BR-1 expressing pleural effusion cells and that this lytic capacity is similar or even above the capacity of Clone2 CAR⁺ T cells (see FIG. 6). The strong activation of pS/MARter electroporated CAR⁺ T cells is also reflected in significantly increased IFNγ secretions compared to mock T cells (see FIG. 5).

### Example 7: Murine 10D11 CAR Expressing T Cells Induce Delayed Tumour Progression and Prolonged Survival Rates in an Allograft Mouse Model

To assess the anti-tumour efficiency of murine 10D11 and Clone2 CARs in vivo, an allograft tumour mouse model was validated using a NY-BR-1 stable expressing murine breast cancer cell line (EO771). Therefore, EO771 cells were seeded in 12 well plates (1×10⁵ cells/well) and cultivated in DMEM (10% FSC, 1% penicillin/streptomycin). The next day, cells were transduced with the lentiviral vector encoding the full-length NY-BR-1 protein and the puromycin resistance gene at an MOI of 5 under the addition of Polybrene (8 μg/mL). 48 h post transduction, medium was exchanged with fresh DMEM. In order to select NY-BR-1+EO771 cells, cells were transferred to a 96 well plate format and cultivated in DMEM medium, supplemented with puromycin (final concentration: 5 μg/mL), over a period of two to three weeks. The NY-BR-1 expression rates were determined with the aid of flow cytometric analyses using the primary Clone2 (3 μg/mL) and secondary APC-conjugated anti-mouse Fcγ subclass I specific antibodies (Jackson Immuno Research), as described in Example 1.

In order to generate the required murine CAR-expressing T cells, spleens were taken from C57BL/6 mice, squeezed through a 100 μm cell strainer (Greiner), washed with PBS (1500 rpm, 5 minutes) and subjected for T cell isolation using the Pan-T cell Isolation Kit II (Miltenyi). Thereafter, murine T cells were cultivated in 12 well plates (5×10⁶ cells/well) with RPMI-1640 medium, supplemented with 10% FCS, 1% penicillin/streptomycin, anti-CD3& antibody (Miltenyi, clone: 145-2C11, 2 μg/mL), anti-CD28 antibody (Miltenyi, clone: 37.51, 1 μg/mL), IL-2 (100 IU/mL), L-glutamine (Gibco, 2 mM), β-mercaptoethanol (PAN Biotech, 50 μM) and non-essential amino acids (Gibco, 2×). 24 h after isolation, murine T cells were electroporated with a DNA plasmid vector including the 10D11 CAR expression cassette comprising SEQ ID NO: 29 or with a DNA plasmid vector including a corresponding Clone2 CAR gene expression cassette together with S/MAR based motifs for episomal maintenance using the Neon Transfection System [ThermoFisher (5×10⁶ T cells and 10 μg DNA per shock)]. Finally, the transfected cells were transferred into 24 well plates with pre-warmed RPMI-1640 medium (5×10⁶ T cells/well), supplemented with 20% FCS, IL-2 (100 IU/mL) and L-glutamine (Gibco, 2 mM). Transfection efficiency was determined by flow cytometric analysis using the PE- or APC-conjugated anti-mouse Fcγ subclass I specific antibodies (Jackson Immuno Research), as described for human CAR⁺ T cells in Example 4.

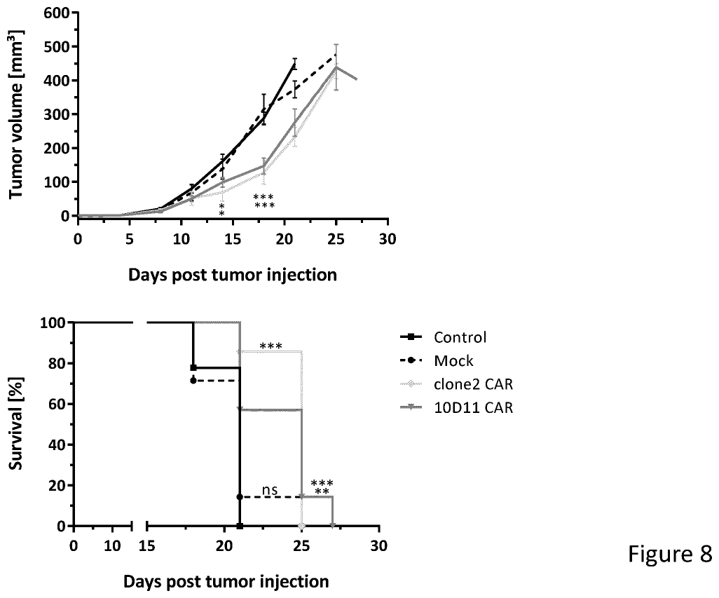

Due to rejection of NY-BR-1+EO771 tumours in immunocompetent C57BL/6 mice (data not shown), 2×10⁶ NY-BR-1+EO771 cells were subcutaneously injected into immunodeficient NOD.CB17-Prkdescid mice. Eight days post tumour engraftment, mice were treated with untransfected (mock) T cells, Clone2 or 10D11 CAR⁺ T cells (5×10⁵ CAR⁺ T cells/mouse) by intravenous injection. Each group, except for control (n=9), consisted of seven mice. Tumour volumes were measured every three to four days and calculated with the ellipse formula (⅙×π×height×width×depth). Tumour engrafted mice were sacrificed at a tumour diameter of 15 mm or a tumour volume of 400 mm³, whichever occurred first. Treatment with T cells treated with T cells positive for the 10D11 CAR of SEQ ID NO:28 and the corresponding Clone2 CAR results in significantly delayed tumour progression compared to mock treated and untreated mice. Furthermore, CAR⁺ T cell treatment provokes significantly prolonged median and overall survival rates compared to untreated mice. In fact, mice treated with T cells positive for the 10D11 CAR of SEQ ID NO:28 even show an increased overall survival rates compared to those treated with Clone2 CAR⁺ T cells. Results are shown in FIG. 8.

### Example 8: NY-BR-1+ Tumour Bearing Mice Show Delayed Tumour Outgrowth and Significantly Extended Overall Survival Rates Through Treatment with Human 10D11 CAR⁺ T Cells

The anti-tumour efficiency of the human 10D11 and Clone2 CARs was further investigated in a xenograft mouse model. First, a stable NY-BR-1 expressing Bosc23 cell line was generated by transduction with the lentiviral vector encoding the NY-BR-1 full-length protein and the puromycin resistance gene under the addition of Polybrene (8 μg/mL), as described for the NY-BR-1+EO771 cell line in Example 7. Following puromycin selection, the remaining clones were tested for NY-BR-1 expression via flow cytometry with the primary Clone2 (3 μg/mL) and secondary APC—conjugated anti-mouse Fcγ subclass I specific antibodies (Jackson Immuno Research), as described in Example 1. The required human CAR expressing T cells were generated through electroporation with DNA plasmid vectors (previously described: in WO 2019/057774, at least on pages 15, 16, 20 and 21; in WO 2019/060253, at least on page 4, line 15 to page 5, line 3; and in “A nonviral, nonintegrating DNA nanovector platform for the safe, rapid, and persistent manufacture of recombinant T cells” (2021; Science Advances, Vol 7, Issue 16), specifically in the section entitled “DNA Vectors” spanning pages 8 and 9 as well as in FIG. 1A, the relevant disclosures of which are incorporated into the present specification by reference) including the 10D11 CAR expression cassette comprising SEQ ID NO:29 or with a corresponding Clone2 CAR gene expression cassette together with the S/MAR motifs, as described in Example 6.

As NOD/SCID/IL2rγ^{null} (NSG) mice lack T, B and NK cells, they allow a better persistence of human lymphocytes and tumour cells compared to NOD.CB17-Prkdc^{scid} mice. Therefore, NSG mice were engrafted with 2×10⁶ NY-BR-1⁺ Bosc23 cell by subcutaneous injection. Eight days post tumour engraftment, untransfected (mock) T cells as well as 10D11 and Clone2 CAR⁺ T cells were injected intravenously (1×10⁶ CAR⁺ T cells/mouse). Untreated mice served as controls. Each group consisted of seven mice. Tumour volumes were measured every three to four days and calculated with the ellipse formula (⅙×π×height×width×depth). Tumour engrafted mice were sacrificed at a tumour diameter of 15 mm or a tumour volume of 400 mm³, whichever occurred first. Indeed, 10D11 CAR⁺ T cell treated mice demonstrate significantly delayed tumour growth, which results in considerable extended overall survival rates compared to mock and untreated mice. Moreover, it is important to note that the Clone2 and 10D11 CAR⁺ T cells do not differ in their anti-tumour efficiency. Results are shown in FIG. 8.

Overall, and consistent with the in vitro experiments, the invented 10D11 scFv-based CAR does not show any deviation from a Clone2-derived CAR in terms of effectiveness and toxicity.

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. An immunoreactive molecule, wherein said immunoreactive molecule specifically recognises and binds to human Ankyrin Repeat Domain-Containing Protein 30A (NY-BR-1; UniProtKB Q9BXX3) characterised in that the immunoreactive molecule specifically recognises and binds to an epitope of NY-BR-1 comprising the antigen peptide sequence of SEQ ID NO:1 and wherein said immunoreactive molecule has an antigen binding region comprising V_{L} complementarity-determining regions (CDRs) 1 to 3 of SEQ ID NOs: 2 to 4 and V_{H} CDRs 1 to 3 of SEQ ID NOs: 5 to 7.

2. The immunoreactive molecule of claim 1, wherein said immunoreactive molecule has an antigen binding region comprising SEQ ID NO:8 and SEQ ID NO:10.

3. The immunoreactive molecule of claim 1, wherein said immunoreactive molecule is a monoclonal mouse antibody, a single-chain variable fragment-fragment crystallisable region (scFv-Fc) fusion protein or a chimeric antigen receptor (CAR).

4. The immunoreactive molecule of claim 1, wherein said immunoreactive molecule is a full-length monoclonal mouse IgG.

5. The immunoreactive molecule of claim 4, wherein said immunoreactive molecule is a full-length monoclonal mouse IgG2a, preferably comprising SEQ ID NOs: 12 and 14.

6. The immunoreactive molecule of claim 1, wherein said immunoreactive molecule is a single-chain variable fragment-fragment crystallisable region (scFv-Fc) fusion protein comprising either a mouse or a human IgG1 Fc domain, preferably comprising either SEQ ID NO:16 or SEQ ID NO:18, respectively.

7. The immunoreactive molecule of claim 1, wherein said immunoreactive molecule is a chimeric antigen receptor (CAR) comprising SEQ ID NO:20.

8. The immunoreactive molecule of claim 7 comprising:
a CD28-derived sequence, preferably SEQ ID NO:22; and/or
a CD3zeta-derived sequence, preferably SEQ ID NO:24; and/or
an OX40-derived sequence, preferably SEQ ID NO:26.

9. The immunoreactive molecule of claim 7 comprising SEQ ID NO. 28.

10. (canceled)

11. A method of treating cancer, said method comprising administering the immunoreactive molecule of claim 1 to a subject in need thereof.

12. (canceled)

13. (canceled)

14. (canceled)

15. (canceled)

16. The method of claim 11, wherein said cancer is characterized by the expression of the cancer associated antigen NY-BR-1.

17. The method of claim 16, wherein said cancer is testicular cancer or breast cancer.

18. A method of detecting the cancer associated antigen NY-BR-1 in a sample, said method comprising the steps of
(a) providing a tissue sample, a primary cell sample or a cell line sample;
(b) contacting said sample with the immunoreactive molecule of claim 1; and
(c) detecting the binding of said immunoreactive molecule to NY-BR-1 in said sample.

19. The method of claim 18, wherein said sample is a sample previously obtained and/or derived from a cancer patient.

20. The method of claim 18, wherein said step of detecting comprises detection of binding of said immunoreactive molecule to NY-BR-1 by immunohistochemical staining; membrane-mediated protein blotting or flow cytometry.
